(19) United States
(12) Patent Application Publication
(10) Pub. No.: US 2017/0004276 A1
(43) Pub. Date: Jan. 5, 2017

(54) BIOMARKER TEST FOR ACUTE CORONARY SYNDROME

(71) Applicants: University of Pittsburgh - Of the Commonwealth System of Higher Education, Pittsburgh, PA (US); Prevencio, Inc., Kirkland, WA (US)

(72) Inventors: William A. LaFramboise, Cheswick, PA (US); Robert V. Masterson, Seattle, WA (US); Oscar C. Marroquin, Pittsburgh, PA (US); Dennis M. McNamara, Venetia, PA (US); Suresh R. Mulukutla, Wexford, PA (US); Aleksey Lomakin, Swapscott, MA (US)

(73) Assignees: University of Pittsburgh - Of the Commonwealth System of Higher Education, Pittsburgh, PA (US); Prevencio, Inc., Kirkland, WA (US)

(21) Appl. No.: 15/265,425

(22) Filed: Sep. 14, 2016

Related U.S. Application Data
(63) Continuation of application No. 13/910,624, filed on Jun. 5, 2013.
Application No. 13/910,624 is a continuation of application No. PCT/US2011/063267, filed on Dec. 5, 2011.
(60) Provisional application No. 61/505,896, filed on Jul. 8, 2011.
(60) Provisional application No. 61/420,158, filed on Dec. 6, 2010.

(51) Int. Cl. G06F 19/00; G01N 33/68

(52) U.S. Cl. CPC G06F 19/3431; G01N 33/6893; G01N 2800/324; G01N 2570/00; G01N 2800/50

(57) ABSTRACT

The present invention relates to biomarker signatures and associated methods for identifying patients that are not likely to manifest significant coronary artery disease. It is based, at least in part, on a study performed on serum samples of 239 patients with clinical symptoms of cardiac distress, some of whom required invasive intervention (stent placement or bypass graft surgery). A set of biomarkers was identified as exhibiting different levels of expression in subjects that did, or did not, require invasive intervention. Further, an algorithm was developed which, using serum levels of these biomarkers, assigned a score to a given patient that was indicative of whether that patient required invasive intervention.

### RELATED APPLICATION INFORMATION

This application is a continuation U.S. patent application Ser. No. 13/910,624, filed Jun. 5, 2013, which is a continuation of International Application Serial No. PCT/US2011/063267, filed Dec. 5, 2011, and claims priority to U.S. Provisional Application Ser. No. 61/505,896, filed on Jul. 8, 2011, and U.S. Provisional Application Ser. No. 61/420,158, filed on Dec. 6, 2010, the contents of which are expressly incorporated by reference herein.

### GRANT INFORMATION

Not applicable.

### 1. INTRODUCTION

The present invention relates to biomarker signatures and associated methods for identifying patients that are not likely to manifest significant coronary artery disease.

### 2. BACKGROUND OF THE INVENTION

In the Western world, cardiovascular disease, typically associated with underlying atherosclerosis, is the leading cause of death (Martin-Ventura et al., 2009, Rev. Esp. Cardiol 62(61:677-688, citing Murray and López, 1997, Lancet 349:1269-1276). Risk factors for cardiac disease are well known, and include hypertension, elevated cholesterol, obesity, and family history. However, despite the prevalence of coronary artery disease (“CAD”) and the appreciation of its risk factors, the link between symptoms and a cardiac event requiring intervention remains elusive. Symptoms can be non-specific—a feeling of heaviness in the chest can reflect CAD but could also be explained by gastric distress; pain in the left arm could be cardiac angina or could be caused by arthritis. Even when pain is known to be cardiac in origin, there can be questions regarding what level of treatment is required; in some scenarios, medication may be sufficient, but in others, surgical intervention is necessary to avoid dire consequences.

A number of technologies have been developed to identify patients at high risk for an adverse cardiac event. Coronary angiography has been considered the “gold standard” but is invasive, costly, and subject to operator-dependent variability (Sharma et al., 2010, Vasc. Health Risk Manag. 6:307-316). Other, less invasive options being explored include coronary computed tomographic angiography (Sharma et al., supra; Cury et al., 2008. J. Nucl. Cardiol. 15(4):564-575), biomarkers (e.g., Martin-Ventura et al., 2009, Rev. Esp. Cardiol 62(6):677-688), adenosine stress magnetic resonance (Ingkanison et al., 2006, J. Am. Coll. Cardiol. 47(7):27:1427-1432), the use of clinical predictors (Tadros et al., 2003. South Med. J. 96(11):1113-1120; Schillinger et al., 2004, Wien Klin. Wochenschr. 116(3):83-89), and indicators of platelet activity (Marcucci et al., 2009, Circulation 119:237-242 (originally published online Dec. 31, 2008); Selvaraj et al., 2004, J. Throm. Thrombolysis 18(2):109-115). There is currently no generally accepted, non-invasive marker for symptomatic CAD that warrants emergent invasive intervention.

### 3. SUMMARY OF THE INVENTION

The present invention relates to biomarker signatures and associated methods for identifying patients that are not likely to manifest significant CAD versus patients that are likely to have significant CAD (and who therefore may benefit from interventional treatment). It is based, at least in part, on a study performed on serum samples of 239 patients with clinical symptoms of cardiac distress or acute coronary syndrome, some of whom required invasive intervention (stent placement or bypass graft surgery). A set of biomarkers was identified that exhibited different levels of expression that discriminated subjects that had significant CAD (and did require invasive intervention) from those patients who did not. Determination of the presence of CAD and the subsequent need for therapeutic invasive intervention was based on performance of a coronary angiography study in each patient. Further, an algorithm was developed using serum levels of these biomarkers to assign a score to each patient that was indicative of whether that patient required invasive intervention. The correlation of the biomarker findings processed by the algorithm to the coronary angiography results indicated that patients without significant CAD could be delineated using the biomarker signatures.

### 4. BRIEF DESCRIPTION OF THE FIGURE

FIG. 1. Receiver-operating characteristics (ROC) for 2 to 5 serum proteins for identification of normal patients with 95% specificity for detection of CAD patients. The ROC curves derived from 4 separate panels were optimized to detect normal patients at highest sensitivity while maintaining specificity of 95% for patients with CAD. The ROC curves are obtained by iteratively testing (100 times) each biomarker panel for classification of a randomly excluded portion (20%) of the dataset. The areas under the curve (AUC) were comparable for 2, 3, 4 and 5 proteins. The sensitivity for detection of normal patients at 95% specificity for CAD patients was 2 proteins=44%, 3 proteins=41%, 4 proteins=50% and 5 proteins=48%.

FIG. 2. Significant differences in apolioprotein B-100, apolipoprotein-A1 and fibrinogen in serum from normal and CAD patients. Solid bars are values expressed as average+standard deviation for apolioprotein B-100 (Apo-B100), apolipoprotein-A1 (Apo-A1) and fibrinogen obtained from normal patients. Open bars are results obtained from patients with coronary artery disease (CAD). Values are expressed in micrograms per milliliter on a logarithmic ordinate scale and each was significantly different (*) between groups (see Table 2).

FIG. 3. Significant differences in vascular cell adhesion molecule, myeloperoxidase, c-reactive protein, resistin and osteopontin in serum from normal and CAD patients. Normal and CAD data is displayed according to FIG. 2 but expressed in nanograms per milliliter on logarithmic ordinate scale. All comparisons represent significant statistical differences delineated in TABLE 7 (*) for vascular cell adhesion molecule (VCAM-1), myeloperoxidase (MPO), c-reactive protein (CRP), resistin and osteopontin (OPN).

FIG. 4. Significant differences in interleukin-6, 1β, 10 and NT-pBNP in serum from normal and CAD patients. Normal and CAD data is displayed according to FIG. 2 but expressed in picograms per milliliter on logarithmic ordinate scale. All comparisons represent significant statistical differences (*) reported in table 2 for interleukin-6 (IL-6), interleukin-1β (IL-1b), interleukin-10 (IL-10) and N-terminal fragment pro-brain natriuretic peptide (NT-pBNP).

FIG. 5. Receiver-operating characteristics (ROC) for 2 to 5 protein panels for identification of normal patients with 95% specificity for detection of CAD patients. The ROC curves are derived from 4 separate panels optimized to detect 101 normal patients (true positives in this figure) at highest specificity while maintaining a sensitivity of 95% for patients with CAD (138 samples). The ROC curves are obtained by iteratively testing each biomarker panel for classification of a randomly excluded portion (20%) of the dataset. The areas under the curve (AUC) were comparable as indicated in the curves for 2 proteins (OPN and resistin: AUC=0.839), 3 proteins (OPN, resistin, apo-B100: AUC=0.845), 4 proteins (IFNg, OPN, MMP-7 and resistin: AUC=0.839) and 5 proteins (IFNg, OPN, MMP-7, resistin and CRP: AUC=0.827). The predicted specificity for detection of normal patients at 95% sensitivity for CAD patients was 2 proteins=50%, 3 proteins=52%, 4 proteins=63% and 5 proteins=64%.

### 5. DETAILED DESCRIPTION OF THE INVENTION

For purposes of clarity, and not by way of limitation, the detailed description of the invention is divided into the following subsections:
(i) biomarker panels;
(ii) diagnostic algorithm;
(iii) kits, and
(iv) methods.

### 5.1 BIOMARKER PANELS

The present invention provides for panels of IT (for “Invasive Treatment”) biomarkers comprising at least two of the following:
osteopontin (“OPN”);
resistin;
interleukin 1β (“IL1b”);
interferon γ (“IFNg”);
myeloperoxidase (“MPO”);
vascular cell adhesion molecule (“VCAM”);
fibrinogen;
matrix metalloproteinase 7 (“MMP7”);
apolipoprotein B100 (“APO-B100”);
C-reactive protein (“CRP”); and
adipocyte complement related protein of 30 kDa (“ACRP30”).

A panel may comprise at least two, at least three, at least four, at least five, at least six, at least seven, at least eight, at least nine, at least ten, or at least eleven IT biomarkers. In non-limiting embodiments of the invention, said panel, in addition to the above-listed IT biomarkers, may include additional biomarkers, for example one additional biomarker, two additional biomarkers, up to five additional biomarkers, up to ten additional biomarkers, up to twenty additional biomarkers, or up to fifty additional biomarkers, where the above-listed IT biomarkers in the panel themselves provide statistically significant information regarding whether a patient would be likely to benefit from invasive CAD intervention and/or would be at increased risk for an adverse cardiac event without further invasive CAD intervention. Accordingly, in non-limiting embodiments, a panel may consist of between 2 and 10, or between 1 and 20, or between 5 and 10, or between 5 and 20, or between 5 and 50 total biomarkers.

In further non-limiting embodiments, a panel may comprise at least two, at least three, at least four, at least five, at least six, at least seven, at least eight, at least nine, at least ten, or at least eleven IT biomarkers as set forth above, and may in addition comprise at least one or at least two or at least three or four of the following serum biomarkers: interleukin 6 (IL-6”), interleukin-10 (“IL-10”), N-terminal pro-brain natriuretic peptide (“NT-pBNP”) and/or apolipoprotein A1 (“Apo-A1”) where IL-6, IL-10 and NT-pBNP serum levels have been observed to increase in subjects having CAD and Apo-A1 has been observed to decrease (see Example sections 7 and 8, below). In specific non-limiting embodiments of the invention, an increase in the serum level of fibrinogen by a factor of 4 or more, and/or an increase in the serum level of VCAM-1, MPO, CRP, resistin and/or osteopontin by a factor of between about 1.3 and 2.5, in consistent with a diagnosis of significant CAD.

Significant CAD is CAD that results in clinical symptoms and/or signs, including one or more of cardiac angina, shortness of breath, diaphoresis, nausea, lightheadedness, palpitations, a positive exercise stress test, ST segment depression by EKG during a standard exercise stress test, and/or stenosis of at least one coronary artery of at least about 70%.

An adverse cardiac event is a decrease in cardiac perfusion that results in permanent damage to the myocardium.

A biomarker is a protein, the serum level of which is associated with a particular biological state. An IT biomarker is a protein, the serum level of which is associated with whether or not a patient would be likely to benefit from invasive CAD intervention and/or would be at increased risk for an adverse cardiac event without further invasive CAD intervention. The predictive value of the expression of an IT biomarker arises when considered together with the expression level of at least one addition IT biomarker identified herein.

In specific, non-limiting embodiments of the invention, the a panel may comprise the following IT biomarkers:
OPN and resistin;
IL1b and OPN;
IFNg and OPN;
OPN and MPO;
OPN, VCAM, and resistin;
OPN, fibrinogen, and resistin;
OPN, MMP7, and resistin;
OPN, resistin, and APO-B100;
OPN, MMP7, VCAM, and resistin;
IFNg, OPN, MMP7 and MPO;
IFNg, OPN, MMP7 and resistin;
OPN, MMP7, resistin and CRP;
IFNg, OPN, MMP7, resistin and CRP; and
IFNg, OPN, MMP7, resistin and ACRP30.

### 5.2 DIAGNOSTIC ALGORITHM

The present invention further provides for an algorithm that may be used to transform the levels of a panel of IT biomarkers, as described above, into a score (hereafter, “IT score” or alternatively, “SF” for scoring function) that may be used to determine whether a patient is not likely to manifest significant CAD.

According to the invention, the following Formula I may be used to compute the IT score:

$$S ( p ) = {A}_{0} + {\sum }_{i = 1}^{N} {A}_{i} Ln ( {M}_{i} ( p ) + {C}_{i} )$$

where S(p) is the IT score for the case p,
constant A₀ is the “Offset Value”
N is the number of markers in the panel
index i lists the markers in the panel
coefficient Aᵢ is the “Coefficient” for the i-th IT biomarker
Mᵢ(p) is the concentration in picograms/ml of the i-th IT biomarker for the case p
Cᵢ is the “LowCutoff” for the i-th marker

The Offset Value (A₀) can be selected to maintain a desired specificity (SP), e.g., 95%, 85%, 80%, etc., and Coefficients (Aᵢ, i=1, . . . N) can be determined utilizing an optimization technique, e.g., a Monte Carlo optimization using a Metropolis algorithm, in order to optimize the sensitivity of Formula I. In some embodiments, the desired specificity can be selected utilizing considerations such as the cost of missing identifying diseased patients and the cost of wrongly diagnosing healthy patients, among others. For a particular level of specificity, e.g., 95%, a “Cutoff Score” (S₀) can be determined by finding the scoring function that provides the best SN at 85% SP and then the cutoff score that provided 95% specificity was determined. When the IT score for a subject S(p) is greater than the Cutoff Score (S₀), subject p is classified as NIT (a patient that would be less likely or unlikely to benefit from invasive CAD intervention and/or would not be at substantially increased risk for an adverse cardiac event without further invasive CAD intervention). When S(p)<S₀, subject p is classified as IT (a patient that would be likely to benefit from invasive CAD intervention and/or would be at increased risk for an adverse cardiac event without further invasive CAD intervention). LowCutoff's (Cᵢ) are included in the formula to reduce “volatility” at low marker values. The value of cᵢ was fixed to 1/10 of each averaged marker value.

In order to determine the performance of Formula I for a given panel of markers, e.g., a panel of 2, 3, 4 or 5 markers, cross-validation can be performed using a select percentage of the data set as a training data set to adjust the Coefficients (Aᵢ), and Cutoffs (Cᵢ). The adjusted Formula I can be tested on another select portion of the data set. In one embodiment involving 24 markers for 101 normal patients and 138 diseased patients, 80% of the data was used as the training data set and the remaining 20% of the data was used to test the specificity and sensitivity of the adjusted scoring function. The cross-validation can be repeated a number of times until the average specificity and sensitivity of the panel are evaluated to the desired accuracy, e.g., approximately 1%. In some embodiments the cross-validation was performed 500 times. The average cross-validated specificity and sensitivity of the panel is an indicator of the panel performance and can be used to rank different panels. Furthermore, the cross-validation can be utilized to determine the optimum panel size. In one embodiment, the performance of panels larger than 3 markers, determined using the cross-validation technique, indicated that those panels were too large for the given size of the sample, e.g., 101 normal patients and 138 diseased patients. Persons of skill in the art will understand that larger data sets can be utilized to identify larger panels, e.g., 4- and 5-marker panels, or even larger, having the desired performance.

In one embodiment, panels of 2, 3, 4 and 5 markers were selected and tested utilizing Formula I for a group of 101 normal patients and 138 patients having CAD. For each panel size, Formula I was used to determine the sensitivity of each panel at 95% specificity and the 50 panels with the highest sensitivity were identified. Cross-validation was then performed on the selected panels, as detailed above, to determine the performance of each panel and rank the panels accordingly.

In the same or another embodiment, artificial markers (e.g., markers having no relation to the disease being investigated) can be introduced into the data set to illustrate the effectiveness of the cross-validation of Formula I. After determining the performance of each panel and ranking the panels, those panels in the top of the final rankings that have artificial markers can be identified. For example, in one embodiment, the top 4 panels that did not include any artificial markers were identified for the 2-, 3-, and 4-marker panels, and the top 2 panels that did not include may artificial markers were identified for the 5-marker panel. The top 4 of the 2-marker panels did not contain any artificial markers; 4 out of 6 of the top 3-marker panels did not contain any artificial markers; 4 out of 6 of the top 4-marker panels did not contain any artificial markers; and 2 out of 38 of the top 5-marker panels did not contain any artificial markers. The presence of an artificial marker in one of the top ranked panels can indicate that the panel's performance is likely coincidental, as a artificial marker by definition cannot predict the likelihood of the target disease.

The present invention provides for a method of determining whether a patient is not likely to manifest significant CAD comprising:
(a) determining the serum levels of at least two biomarkers selected from the group consisting of OPN, resistin, IL1b, IFNg, MPO, VCAM, fibrinogen, MMP7, APO-B100, CRP, ACRP30;
(b) determining an Offset Value for the combination of biomarkers measured in (a);
(c) determining Coefficients for each of the biomarkers measured in (a);
(d) using the following Formula I to transform the serum levels determined in (a) into an IT score;

$$S ( p ) = {A}_{0} + {\sum }_{i = 1}^{N} {A}_{i} Ln ( {M}_{i} ( p ) + {C}_{i} )$$

where S(p) is the IT score for the case p,
constant A₀ is the “Offset Value”
N is the number of markers in the panel
index i lists the markers in the panel
coefficient Aᵢ is the “Coefficient” for the i-th IT biomarker
Mᵢ(p) is the concentration in picograms/ml of the i-th IT biomarker for the case p
Cᵢ is the “LowCutoff” for the i-th marker
(LowCutOff's (Cᵢ) are included to reduce “volatility” at low marker values. The exact values of Cᵢ is fixed to 1/10 of the averaged marker value.) and
(e) comparing the IT score to a Cutoff Score,
where an IT score greater than the Cutoff Score indicates that the patient is not likely to manifest significant and an IT score less than the Cutoff Score indicates that the patient is likely to manifest significant CAD and could benefit from invasive intervention.

In specific, non-limiting embodiments of the invention, Offset Values, Coefficients, and Cutoff Scores that may be used for calculating, using Formula I, and interpreting an IT score for a patient based on any of 14 different panels of IT biomarkers are set forth in TABLE 2, below.

Accordingly, in one specific, non-limiting embodiment, the present invention provides for a method of determining whether a patient is not likely to manifest significant CAD comprising:
(a) determining the serum levels of OPN and resistin;
(b) using the following Formula I to transform the serum levels determined in (a) into an IT score (S(p));

$$S ( p ) = {A}_{0} + {\sum }_{i = 1}^{N} {A}_{i} Ln ( {M}_{i} ( p ) + {C}_{i} )$$

wherein the Offset Value A₀ is 16.35,

### N=2,

index i lists the markers in the panel,
the Coefficient A₁ for OPN is −0.70, and the Coefficient A₂ for resistin is −0.82,
Mᵢ(p) is the concentration in picograms/ml of the i-th IT biomarker for the patient, and the Low Cutoff C₁ for OPN is 3533 and C₂ for resistin is 9378.3;
where an IT score greater than 0.47 indicates that the patient is not likely to manifest significant CAD and an IT score less than 0.47 indicates that, at 95% specificity, the patient is likely to manifest significant CAD and could benefit from invasive intervention.

In another specific, non-limiting embodiment, the present invention provides for a method of determining whether a patient is not likely to manifest significant CAD comprising:
(a) determining the serum levels of IL1b and OPN;
(b) using the following Formula I to transform the serum levels determined in (a) into an IT score (S(p));

$$S ( p ) = {A}_{0} + {\sum }_{i = 1}^{N} {A}_{i} Ln ( {M}_{i} ( p ) + {C}_{i} )$$

wherein the Offset Value A₀ is 14.66,

### N=2,

index i lists the markers in the panel,
the Coefficient A₁ for IL1b is −1.12, and the Coefficient A₂ for OPN is −1.06,
Mᵢ(p) is the concentration in picograms/ml of the i-th IT biomarker for the patient, and

### the Low Cutoff C, for IL1b is 8.6 and C₂ for OPN is 3533;

where an IT score greater than 1.39 indicates that the patient is not likely to manifest significant CAD and an IT score less than 1.39 indicates that, at 95% specificity, the patient is likely to manifest significant CAD and could benefit from invasive intervention.

In another specific, non-limiting embodiment, the present invention provides for a method of determining whether a patient is not likely to manifest significant CAD comprising:
(a) determining the serum levels of IFNg and OPN;
(b) using the following Formula I to transform the serum levels determined in (a) into an IT score (S(p));

$$S ( p ) = {A}_{0} + {\sum }_{i = 1}^{N} {A}_{i} Ln ( {M}_{i} ( p ) + {C}_{i} )$$

wherein the Offset Value A₀ is 11.91,

### N=2,

index i lists the markers in the panel,
the Coefficient A₁ for IFNg is −0.60, and the Coefficient A₂ for OPN is −1.14,
Mᵢ(p) is the concentration in picograms/ml of the i-th IT biomarker for the patient, and the Low Cutoff C₁ for IFNg is 0.4123 and C₂ for OPN is 3533;
where an IT score greater than 0.97 indicates that the patient is not likely to manifest significant CAD and an IT score less than 0.97 indicates that, at 95% specificity, the patient is likely to manifest significant CAD and could benefit from invasive intervention.

In another specific, non-limiting embodiment, the present invention provides for a method of determining whether a patient is not likely to manifest significant CAD comprising:
(a) determining the serum levels of OPN and MPO;
(b) using the following Formula I to transform the serum levels determined in (a) into an IT score (S(p));

$$S ( p ) = {A}_{0} + {\sum }_{i = 1}^{N} {A}_{i} Ln ( {M}_{i} ( p ) + {C}_{i} )$$

wherein the Offset Value A₀ is 16.28,

### N=2,

index i lists the markers in the panel,
the Coefficient A₁ for OPN is −0.68, and the Coefficient A₂ for MPO is −0.72,
Mᵢ(p) is the concentration in picograms/ml of the i-th IT biomarker for the patient, and
the Low Cutoff C₁ for OPN is 3533 and C₂ for MPO is 54192;
where an IT score greater than 0.57 indicates that the patient is not likely to manifest significant CAD and an IT score less than 0.57 indicates that, at 95% specificity, the patient is likely to manifest significant CAD and could benefit from invasive intervention.

In another specific, non-limiting embodiment, the present invention provides for a method of determining whether a patient is not likely to manifest significant CAD comprising:
(a) determining the serum levels of OPN, VCAM and resistin;
(b) using the following Formula I to transform the serum levels determined in (a) into an IT score (S(p));

$$S ( p ) = {A}_{0} + {\sum }_{i = 1}^{N} {A}_{i} Ln ( {M}_{i} ( p ) + {C}_{i} )$$

wherein the Offset Value A₀ is 19.72,

### N=3,

index i lists the markers in the panel,
the Coefficient A₁ for OPN is −0.60, the Coefficient A₂ for VCAM is −0.37 and the Coefficient A₃ for resistin is −0.75,
Mᵢ(p) is the concentration in picograms/ml of the i-th IT biomarker for the patient, and the Low Cutoff C₁ for OPN is 3533, C₂ for VCAM is 102448 and C₃ for resistin is 9378.3;
where an IT score greater than 0.44 indicates that the patient is not likely to manifest significant CAD and an IT score less than 0.44 indicates that, at 95% specificity, the patient is likely to manifest significant CAD and could benefit from invasive intervention.

In another specific, non-limiting embodiment, the present invention provides for a method of determining whether a patient is not likely to manifest significant CAD comprising:
(a) determining the serum levels of OPN, fibrinogen and resistin;
(b) using the following Formula I to transform the serum levels determined in (a) into an IT score (S(p));

$$S ( p ) = {A}_{0} + {\sum }_{i = 1}^{N} {A}_{i} Ln ( {M}_{i} ( p ) + {C}_{i} )$$

wherein the Offset Value A₀ is 19.38,

### N=3,

index i lists the markers in the panel,
the Coefficient A₁ for OPN is −0.60, the Coefficient A₂ for fibrinogen is −0.28 and the Coefficient A₃ for resistin is −0.79,
Mᵢ(p) is the concentration in picograms/ml of the i-th IT biomarker for the patient, and
the Low Cutoff C₁ for OPN is 3533, C₂ for fibrinogen is 1236057 and C₃ for resistin is 9378.3;
where an IT score greater than 0.5 indicates that the patient is not likely to manifest significant CAD and an IT score less than 0.5 indicates that, at 95% specificity, the patient is likely to manifest significant CAD and could benefit from invasive intervention.

In another specific, non-limiting embodiment, the present invention provides for a method of determining whether a patient is not likely to manifest significant CAD comprising:
(a) determining the serum levels of OPN, MMP7 and resistin;
(b) using the following Formula I to transform the serum levels determined in (a) into an IT score (S(p));

$$S ( p ) = {A}_{0} + {\sum }_{i = 1}^{N} {A}_{i} Ln ( {M}_{i} ( p ) + {C}_{i} )$$

wherein the Offset Value A₀ is 18.96,

### N=3,

index i lists the markers in the panel,
the Coefficient A₁ for OPN is −0.74, the Coefficient A₂ for MMP7 is −0.36, and the Coefficient A₃ for resistin is −0.74,
Mᵢ(p) is the concentration in picograms/ml of the i-th IT biomarker for the patient, and the Low Cutoff C₁ for OPN is 3533, C₂ for MMP7 is 519.89 and C₃ for resistin is 9378.3;
where an IT score greater than 0.49 indicates that the patient is not likely to manifest significant CAD and an IT score less than 0.49 indicates that, at 95% specificity, the patient is likely to manifest significant CAD and could benefit from invasive intervention.

In another specific, non-limiting embodiment, the present invention provides for a method of determining whether a patient is not likely to manifest significant CAD comprising:
(a) determining the serum levels of OPN, resistin, and APO-B100;
(b) using the following Formula I to transform the serum levels determined in (a) into an IT score (S(p));

$$S ( p ) = {A}_{0} + {\sum }_{i = 1}^{N} {A}_{i} Ln ( {M}_{i} ( p ) + {C}_{i} )$$

wherein the Offset Value A₀ is 19.88,

### N=3,

index i lists the markers in the panel,
the Coefficient A₁ for OPN is −0.50, the Coefficient A₂ for resistin is −0.57, and the Coefficient A₃ for APO-B100 is −0.42,
Mᵢ(p) is the concentration in picograms/ml of the i-th IT biomarker for the patient, and
the Low Cutoff C₁ for OPN is 3533, C₂ for resistin is 9378.3 and C₃ for APO-B100 is 32404058;
where an IT score greater than 0.35 indicates that the patient is not likely to manifest significant CAD and an IT score less than 0.35 indicates that, at 95% specificity, the patient is likely to manifest significant CAD and could benefit from invasive intervention.

In another specific, non-limiting embodiment, the present invention provides for a method of determining whether a patient is not likely to manifest significant CAD comprising:
(a) determining the serum levels of OPN, MMP7, VCAM and resistin;
(b) using the following Formula I to transform the serum levels determined in (a) into an IT score (S(p));

$$S ( p ) = {A}_{0} + {\sum }_{i = 1}^{N} {A}_{i} Ln ( {M}_{i} ( p ) + {C}_{i} )$$

wherein the Offset Value A₀ is 21.98,

### N=4,

index i lists the markers in the panel,
the Coefficient A₁ for OPN is −0.60, the Coefficient A₂ for MMP7 is −0.37, the Coefficient A₃ for VCAM is −0.32 and the Coefficient A₄ for resistin is −0.73,
Mᵢ(p) is the concentration in picograms/ml of the i-th IT biomarker for the patient, and
the Low Cutoff C₁ for OPN is 3533, C₂ for MMP7 is 519.89, C₃ for VCAM is 102448, and C₄ for resistin is 9378.3;
where an IT score greater than 0.41 indicates that the patient is not likely to manifest significant CAD and an IT score less than 0.41 indicates that, at 95% specificity, the patient is likely to manifest significant CAD and could benefit from invasive intervention.

In another specific, non-limiting embodiment, the present invention provides for a method of determining whether a patient is not likely to manifest significant CAD comprising:
(a) determining the serum levels of IFNg, OPN, MMP7 and MPO;
(b) using the following Formula I to transform the serum levels determined in (a) into an IT score (S(p));

$$S ( p ) = {A}_{0} + {\sum }_{i = 1}^{N} {A}_{i} Ln ( {M}_{i} ( p ) + {C}_{i} )$$

wherein the Offset Value A₀ is 19.05,

### N=4,

index i lists the markers in the panel,
the Coefficient A₁ for IFNg is −0.38, the Coefficient A₂ for OPN is −0.80, the Coefficient A₃ for MMP7 is −0.35, and the Coefficient A₄ for MPO is −0.59,
Mᵢ(p) is the concentration in picograms/ml of the i-th IT biomarker for the patient, and the Low Cutoff C₁ for IFNg is 0.4123, C₂ for OPN is 3533, C₃ for MMP7 is 519.89 and C₄ for MPO is 54192;
where an IT score greater than 0.56 indicates that the patient is not likely to manifest significant CAD and an IT score less than 0.56 indicates that, at 95% specificity, the patient is likely to manifest significant CAD and could benefit from invasive intervention.

In another specific, non-limiting embodiment, the present invention provides for a method of determining whether a patient is not likely to manifest significant CAD comprising:
(a) determining the serum levels of IFNg, OPN, MMP7, and resistin;
(b) using the following Formula I to transform the serum levels determined in (a) into an IT score (S(p));

$$S ( p ) = {A}_{0} + {\sum }_{i = 1}^{N} {A}_{i} Ln ( {M}_{i} ( p ) + {C}_{i} )$$

wherein the Offset Value A₀ is 19.57,

### N=4,

index i lists the markers in the panel,
the Coefficient A₁ for IFNg is −0.22, the Coefficient A₂ for OPN is −0.88, the Coefficient A₃ for MMP7 is −0.61 and the Coefficient A₄ for resistin is −0.47,
Mᵢ(p) is the concentration in picograms/ml of the i-th IT biomarker for the patient, and
the Low Cutoff C₁ for IFNg is 0.4123, C₂ for OPN is 3533, C₃ for MMP7 is 519.89, and C₄ for resistin is 9378.3;
where an IT score greater than 0.58 indicates that the patient is not likely to manifest significant CAD and an IT score less than 0.58 indicates that, at 95% specificity, the patient is likely to manifest significant CAD and could benefit from invasive intervention.

In another specific, non-limiting embodiment, the present invention provides for a method of determining whether a patient is not likely to manifest significant CAD comprising:
(a) determining the serum levels of OPN, MMP7, resistin and CRP;
(b) using the following Formula I to transform the serum levels determined in (a) into an IT score (S(p));

$$S ( p ) = {A}_{0} + {\sum }_{i = 1}^{N} {A}_{i} Ln ( {M}_{i} ( p ) + {C}_{i} )$$

wherein the Offset Value A₀ is 22.50,

### N=4,

index i lists the markers in the panel,
the Coefficient A₁ for OPN is −0.69, the Coefficient A₂ for MMP7 is −0.73, the Coefficient A₃ for resistin is −0.55 and the Coefficient A₄ for CRP is −0.25,
Mᵢ(p) is the concentration in picograms/ml of the i-th IT biomarker for the patient, and
the Low Cutoff C₁ for OPN is 3533, C₂ for MMP7 is 519.89, C₃ for resistin is 9378.3 and C₄ for CRP is 51754;
where an IT score greater than 0.56 indicates that the patient is not likely to manifest significant CAD and an IT score less than 0.56 indicates that, at 95% specificity, the patient is likely to manifest significant CAD and could benefit from invasive intervention.

In another specific, non-limiting embodiment, the present invention provides for a method of determining whether a patient is not likely to manifest significant CAD comprising:
(a) determining the serum levels of IFNg, OPN, MMP7, resistin and CRP;
(b) using the following Formula I to transform the serum levels determined in (a) into an IT score (S(p));

$$S ( p ) = {A}_{0} + {\sum }_{i = 1}^{N} {A}_{i} Ln ( {M}_{i} ( p ) + {C}_{i} )$$

wherein the Offset Value A₀ is 21.77,

### N=5,

index i lists the markers in the panel,
the Coefficient A₁ for IFNg is −0.18, the Coefficient A₂ for OPN is −0.74, the Coefficient A₃ for MMP7 is −0.76, the Coefficient A₄ for resistin is −0.50 and the Coefficient A₅ for CRP is −0.16,
Mᵢ(p) is the concentration in picograms/ml of the i-th IT biomarker for the patient, and the Low Cutoff C₁ for IFNg is 0.4123, C₂ for OPN is 3533, C₃ for MMP7 is 519.89, C₄ for resistin is 9378.3, C₅ for CRP is 51754;
where an IT score greater than 0.47 indicates that the patient is not likely to manifest significant CAD and an IT score less than 0.47 indicates that, at 95% specificity, the patient is likely to manifest significant CAD and could benefit from invasive intervention.

In another specific, non-limiting embodiment, the present invention provides for a method of determining whether a patient is not likely to manifest significant CAD comprising:
(a) determining the serum levels of IFNg, OPN, MMP7, resistin and ACRP30;
(b) using the following Formula I to transform the serum levels determined in (a) into an IT score (S(p));

$$S ( p ) = {A}_{0} + {\sum }_{i = 1}^{N} {A}_{i} Ln ( {M}_{i} ( p ) + {C}_{i} )$$

wherein the Offset Value A₀ is 11.41,

### N=5,

index i lists the markers in the panel,
the Coefficient A₁ for IFNg is −0.35, the Coefficient A₂ for OPN is −0.82, the Coefficient A₃ for MMP7 is −0.22, the Coefficient A₄ for resistin is −0.53 and the Coefficient A₅ for ACRP30 is 0.33,
Mᵢ(p) is the concentration in picograms/ml of the i-th IT biomarker for the patient, and the Low Cutoff C₁ for IFNg is 0.4123, C₂ for OPN is 3533, C₃ for MMP7 is 519.89, C₄ for resistin is 9378.3, C₅ for ACRP30 is 493266;
where an IT score greater than 0.61 indicates that the patient is not likely to manifest significant CAD and an IT score less than 0.61 indicates that, at 95% specificity, the patient is likely to manifest significant CAD and could benefit from invasive intervention.

### 5.3 KITS

The present invention provides for kits for detecting the IT biomarkers discussed above. Such kits may comprise a means for measuring the serum levels of a panel of biomarkers comprising at least two IT biomarkers selected from the group consisting of OPN, resistin, IL1b, IFNg, MPO, VCAM, fibrinogen, MMP7, APO-B100, CRP, ACRP30. Said kit may optionally further comprise a means for measuring the serum levels of a serum biomarker selected from the group consisting of IL-6, IL-10, NT-pBNP and Apo-A1. In non-limiting embodiments, a panel may consist of between 2 and 10, or between 1 and 20, or between 5 and 10, or between 5 and 20, or between 5 and 50 total biomarkers. Means for measuring such serum levels are known in the art, and include, for example, the use of a capture agent, which optionally is detectably labeled, where the capture agent may be used together with a detection agent that binds to the biomarker and/or the capture agent. A capture agent may be, for example and not by limitation, an antibody, a portion of an antibody such as a Fab or Fab2 fragment, a single chain antibody, a receptor for the biomarker or a portion thereof or a ligand for the biomarker or a portion thereof. Likewise, a detection agent may be, for example and not by limitation, an antibody, a portion of an antibody such as a Fab or Fab2 fragment, a single chain antibody, a receptor for the biomarker or capture agent or a portion thereof or a ligand for the biomarker or capture agent or a portion thereof. The capture agent and/or detection agent may be detectably labeled using a radioactive label, a fluorescent label, a chemical label, an oligonucleotide label, an enzymatic label, or a protein label (e.g. a fluorescent protein such as Green Fluorescent Protein). Standard techniques that may be used, for example, include enzyme-linked immunoabsorbent assay (“ELISA”) or Western blot.

In addition to the methods described above, any method known in the art for quantitatively measuring levels of protein in a sample, e.g., non-antibody based methods, can be used in the methods and kits of the invention. For example, mass spectrometry-based (such as, for example, Multiple Reaction Monitoring (MRM) mass spectrometry) or HPLC-based methods can be used. Methods of protein quantification are described in, for example, Ling-Na Zheng et al., 2011, J. of Analytical Atomic Spectrometry, 26, 1233-1236; Vaudel, M., et al., 2010, Proteomics, Vol. 10: 4; Pan, S., 2009 J. Proteome Research, February; 8(2):787-97; Westermeier and Marouga, 2005, Bioscience Reports, Vol. 25, Nos. 1/2; Carr and Anderson, 2008, Clinical Chemistry. 54:1749-1752; and Aebersold and Mann, 2003, Nature, Vol. 422.

Additional, more recent technologies, such as those used in the field of proteomics, may be embodied in kits of the invention. Such technologies include the use of microfluidic chips and related technologies as described, for example, in United States Patent Application No. US 2008/0202927; Sorger, 2008, Nature Biotechnol. 26:1345-1346; Li et al., 2002, Mol. Cell. Proteomics 1.2:157; Hou et al., 2006, J. Proteome Res. 5(10):2754-2759; Li et al., 2001, Proteomics 1(8):975-986; Ramsey et al., 2003, Anal. Chem. 75(15):3758-3764; Armenta et al., 2009, Electrophoresis 30(7): 1145-1156; Lynch et al., 2004, Proteomics 4(6):1695-1702; Kingsmore et al., 2003, Curr. Opin. Biotechnol. 14(1):74-81).

In non-limiting embodiments of the invention, a kit provides a means for measuring serum levels of a panel of biomarkers comprising one of the following combinations of IT biomarkers:
OPN and resistin;
IL1b and OPN;
IFNg and OPN;
OPN and MPO;
OPN, VCAM, and resistin;
OPN, fibrinogen, and resistin;
OPN, MMP7, and resistin;
OPN, resistin, and APO-B100;
OPN, MMP7, VCAM, and resistin;
IFNg, OPN, MMP7 and MPO;
IFNg, OPN, MMP7 and resistin;
OPN, MMP7, resistin and CRP;
IFNg, OPN, MMP7, resistin and CRP; and
IFNg, OPN, MMP7, resistin and ACRP30
   In specific, non-limiting examples, the means for detection is an antibody or variable-region containing fragment thereof that binds to the IT biomarker, where said antibody or fragment is either directly or indirectly detectably labeled; an indirect label may be a second antibody or a labeled version of the IT biomarker, as are known in the art.

In non-limiting embodiments of the invention, a kit may further comprise software that (i) determines or assigns an Offset Value for the combination of IT biomarkers used in the kit; (ii) determines or assigns Coefficients for each of the IT biomarkers used in the kit; (iii) uses the following Formula I to transform the serum levels determined using the kit into an IT score;

$$S ( p ) = {A}_{0} + {\sum }_{i = 1}^{N} {A}_{i} Ln ( {M}_{i} ( p ) + {C}_{i} )$$

where S(p) is the IT score for the case p,
constant A₀ is the “Offset Value”
N is the number of markers in the panel
index i lists the markers in the panel
coefficient Aᵢ is the “Coefficient” for the i-th IT biomarker
Mᵢ(p) is the concentration in picograms/ml of the i-th IT biomarker for the case p
Cᵢ is the “LowCutoff” for the i-th marker and

(iv) compares the IT score to a Cutoff Score,

where an IT score greater than the Cutoff Score indicates that the patient would not be at increased risk for an adverse cardiac event without further invasive coronary artery disease intervention and an IT score less than the Cutoff Score indicates that the patient would be at increased risk for an adverse cardiac event without further invasive coronary artery disease intervention. In specific, non-limiting embodiments of the invention, Offset Values, Coefficients, and Cutoff Scores that may be used by this software for calculating, using Formula I, and interpreting an IT score for a patient based on any of 14 different panels of IT biomarkers are set forth in TABLE 2, below.

### 5.4 METHODS

The present invention provides for a method of treating a patient suffering from one or more symptom consistent with coronary artery disease, including but not limited to, one or more of chest pain, chin pain, shoulder pain, arm pain, shortness of breath, diaphoresis, weakness, and nausea, comprising performing the diagnostic method set forth above, and, where the IT score indicates that the patient is likely to manifest significant CAD (and therefore may be at increased risk for an adverse cardiac event without further invasive coronary artery disease intervention), recommending, to the patient, an invasive CAD intervention procedure. Suitable invasive CAD intervention procedures include, but are not limited to, one or more of stent placement, balloon dilatation, laser angioplasty, rotary atherectomy, bypass graft placement, and pacemaker placement. The present invention also provides for the further step of performing the procedure.

In related embodiments, the present invention provides for a method of treating a patient suffering from one or more symptom consistent with coronary artery disease, including but not limited to, one or more of chest pain, chin pain, shoulder pain, arm pain, shortness of breath, diaphoresis, weakness, and nausea, comprising determining whether, in the patient, the serum level of one or two or three or four or five or six or seven or eight or nine or ten or eleven of the following proteins is elevated: Apo-B100, fibrinogen, VCAM-1, myeloperoxidase, CRP, resistin, osteopontin, IL-6, IL-1b, IL-10 and NT-pBNP and/or whether the level of Apo-A1 is decreased, where said increase and/or decrease is consistent with the patient having significant CAD. Such methods may be performed independently or in conjunction with generation of the IT score as set forth above. If the results are consistent with the patient having significant CAD, an invasive CAD intervention procedure may be recommended or performed.

In some embodiments, the methods of the present invention are used in conjunction with one or more additional clinical scoring system in order to determine the appropriate clinical course of action for a patient. For example, where the methods described above are carried out and the results indicate that the patient does not have CAD requiring invasive CAD intervention, but the patient does have other risk factors for CAD (for example, advanced age or obesity) or additional health issues, a physician may consider the overall risk and recommend or perform an invasive CAD intervention procedure.

### 6. EXAMPLE

### Identification of Cad Biomarkers

Serum samples were evaluated from 239 patients with clinical symptoms of cardiac stress. All underwent coronary angiography studies for diagnosis of coronary artery disease (CAD). One hundred and thirty eight of these patients required subsequent medical therapy for CAD i.e. percutaneous intervention (PCI) comprising placement of a stent or coronary artery bypass graft surgery. These patients are referred to as the P group. All serum specimens were assayed using a multiplex, sandwich ELISA protocol targeting 24 proteins through the use of monoclonal capture antibodies spotted in 96 well microplates. A second level of specificity is obtained in the assay after initial target capture through the use of a different set of monoclonal detection antibodies labeled with a fluorescent marker. The results are presented in TABLE 1 with mean concentration values in picograms per milliliter for the P group (PCI) versus the N group of 101 subjects who did not undergo percutaneous intervention for CAD. The proteins are ranked according to lowest p values derived from the standard students unpaired T test. Seven analytes were statistically significant after Bonferoni correction for 24 targets (0.05/n:24=0.0020) for values from P versus N subjects.

TABLE 1

| PROTEIN | Mean N | Std DeV | Mean P | Std Dev | P value | PROTEIN |
|---|---|---|---|---|---|---|
| OPN | 16227.3 | 15964.3 | 49567.6 | 40913.3 | 2.47E−13 | OPN |
| Apo-A1 | 3.01E+08 | 2.59E+08 | 1.54E+08 | 1.35E+08 | 3.93E−08 | Apo-A1 |
| VCAM | 8.38E+05 | 3.69E+05 | 1.17E+06 | 4.99E+05 | 5.08E−08 | VCAM |
| Apo-B100 | 3.01E+08 | 8.26E+07 | 3.43E+08 | 7.87E+07 | 5.53E−05 | Apo-B100 |
| MPO | 4.46E+05 | 2.64E+05 | 6.16E+05 | 3.69E+05 | 9.41E−05 | MPO |
| IL1b | 48.0 | 117.7 | 113.7 | 168.8 | 0.0009 | IL1b |
| CRP | 2.75E+05 | 5.49E+05 | 6.99E+05 | 1.24E+06 | 0.0016 | CRP |
| NT-pBNP | 41.1 | 111.1 | 101.7 | 202.5 | 0.0070 | NT-pBNP |
| Resistin | 8.10E+04 | 6.20E+04 | 1.04E+05 | 7.00E+04 | 0.0094 | Resistin |
| Fibrinogen | 3.95E+06 | 6.27E+06 | 1.86E+07 | 5.91E+07 | 0.0139 | Fibrinogen |
| IL6 | 578.3 | 1135.9 | 942.3 | 1154.5 | 0.0163 | IL6 |
| IL10 | 3.2 | 3.5 | 7.5 | 18.2 | 0.0183 | IL10 |
| MMP1 | 4751.2 | 2249.5 | 5341.6 | 2404.8 | 0.0541 | MMP1 |
| MMP7 | 4896.7 | 3018.6 | 5457.7 | 2408.6 | 0.1106 | MMP7 |
| Leptin | 13941.2 | 17598.7 | 10612.3 | 15495.5 | 0.1232 | Leptin |
| TNFa | 22.4 | 73.4 | 15.0 | 19.4 | 0.2603 | TNFa |
| L-Selectin | 1.15E+06 | 2.99E+05 | 1.11E+06 | 2.71E+05 | 0.3189 | L-Selectin |
| Acrp30 | 5.21E+06 | 3.86E+06 | 4.77E+06 | 3.46E+06 | 0.3536 | Acrp30 |
| PECAM-1 | 32059.6 | 28990.9 | 35035.6 | 25334.5 | 0.3997 | PECAM-1 |
| TIMP1 | 3.20E+05 | 1.15E+05 | 3.29E+05 | 8.79E+04 | 0.4804 | TIMP1 |
| MCP1 | 3069.1 | 3518.0 | 3347.3 | 3418.7 | 0.5402 | MCP1 |
| TM | 1444.0 | 864.4 | 1391.7 | 444.4 | 0.5414 | TM |
| IFNg | 3.9 | 12.9 | 4.3 | 7.7 | 0.7831 | IFNg |
| E-Selectin | 33942.6 | 19238.4 | 34308.7 | 16525.3 | 0.8743 | E-Selectin |

In TABLE 1, the column labeled Protein indicates the markers tested in the patient serum groups including OPN: osteopontin, Apo-A1: apolipoprotein A1, VCAM: vascular cell adhesion molecule, Apo-B100: apolipoprotein B100, MPO: myeloperoxidase, IL1b: interleukin 1 beta, CRP: c reactive protein, NT-pBNP: N-terminal pro-brain natriuretic peptide, Resistin, Fibrinogen, IL6: interleukin 6, IL10: interleukin 10, MMP1: matrix metalloproteinase 1, MMP7: matrix metalloproteinase 7, leptin, TNFa: tumor necrosis factor alpha, L-Selectin: leukocyte cell adhesion molecule 1, Acrp30: adipocyte complement related protein of 30 kDa, PECAM-1: platelet endothelial cell adhesion molecule 1, TIMP1: tissue inhibitor of metalloproteinase's 1, MCP1: monocyte chemotactic protein 1, TM: tropomodulin, IFNg: interferon gamma, E-Selectin: endothelial adhesion molecule 1. Mean and standard deviations are reported in picograms/ml for the N group (n=101 samples) and the P group (n=138 samples) and p values were obtained by the students T test for unpaired values. Values less than p<0.002 were considered significant based on the Bonferroni correction for multiple tests and are highlighted.

These 24 markers were evaluated on a post hoc basis to select biomarker signatures for their ability to discriminate patients from the P group (those that underwent PCI) versus the N group (no PCI). Multiple marker panels were delineated composed of 2, 3, 4 or 5 markers based on the highest sensitivity to classify members of the N group (SN) while maintaining high specificity for the P group (SP) i.e. SP was fixed at 95% yielding only 7 misclassified P cases out of the 138 total. The final selection of the panels was based on best performance at SP=95% with the additional requirement that predictive performance was also good at 85% and 90% SP. Preliminary evaluation of panels was performed at SP=85%, a condition providing approximately equal numbers of misclassified P and N cases as well as providing the best condition for selecting panels differentiating between P and N.

For each biomarker panel a scoring function (SF) was defined that provided the best SN at 85% SP and then a cutoff score that provided SP=95% was determined. Panels were then cross-validated by randomly excluding 20% of cases from the training set, optimizing the SF for the remaining 80% of cases and using the SF to diagnose the excluded cases. The cross-validation procedure was repeated 100 times, the average ROC curve was accumulated, and the area under curve (AUC) and sensitivities at SP=90%, 95% and 98% were determined.

The analytical form of the algorithm used to compute the scoring (SF) (“IT Score”) is:

$$S ( p ) = {A}_{0} + {\sum }_{i = 1}^{N} {A}_{i} Ln ( {M}_{i} ( p ) + {C}_{i} )$$

S(p) is the score for the case p,
constant A₀ is the “Offset”
N is the number of markers in the panel
index i lists the markers in the panel
coefficient Aᵢ is the “Coefficient” for the i-th marker
Mᵢ(p) is the concentration in picograms/ml of the i-th marker for the case p
Cᵢ is the “LowCutoff” for the i-th marker

The optimization algorithm determined the Offset (A₀) and Coefficients (Aᵢ, i=1, . . . N) that provided scores that best discriminated between P and N groups. For a specific SP, e.g. 95%, a “Cutoff” S₀ can be determined. When S(p)>S₀, case p is classified as N. When S(p)<S₀, case p is classified as P. LowCutoff's (C₁) are included in the formula to reduce “volatility” at low marker values. The value of c₁ was fixed to 1/10 of each averaged marker value.

The scoring function (SF) algorithm delineated a series of marker panels comprising 2 to 5 individual markers, with the ability to discriminate N from P values with increasing sensitivity. Twelve individual markers in 14 combinations were represented in these marker panels. The coefficient for each marker within each biomarker panel and its offset value is displayed in TABLE 2.

TABLE 2

| | Offset | | Coef | | Coef | | Coef | | Coef | | Coef |
|---|---|---|---|---|---|---|---|---|---|---|---|
| | Ao | Marker | Ai | Marker | Ai | Marker | Ai | Marker | Ai | Marker | Ai |
| 1 | 16.35 | OPN | −0.70 | Resistin | −0.82 | | | | | | |
| 2 | 14.66 | IL1b | −1.12 | OPN | −1.06 | | | | | | |
| 3 | 11.91 | IFNg | −0.60 | OPN | −1.14 | | | | | | |
| 3 | 16.28 | OPN | −0.68 | MPO | −0.72 | | | | | | |
| 5 | 19.72 | OPN | −0.60 | VCAM | −0.37 | Resistin | −0.75 | | | | |
| 6 | 19.38 | OPN | −0.60 | Fibrinogen | −0.28 | Resistin | −0.79 | | | | |
| 7 | 18.96 | OPN | −0.74 | MMP7 | −0.36 | Resistin | −0.74 | | | | |
| 8 | 19.88 | OPN | −0.50 | Resistin | −0.57 | Apo-B100 | −0.42 | | | | |
| 9 | 21.98 | OPN | −0.60 | MMP7 | −0.37 | VCAM | −0.32 | Resistin | −0.73 | | |
| 10 | 19.05 | IFNg | −0.38 | OPN | −0.80 | MMP7 | −0.35 | MPO | −0.59 | | |
| 11 | 19.57 | IFNg | −0.22 | OPN | −0.88 | MMP7 | −0.61 | Resistin | −0.47 | | |
| 12 | 22.50 | OPN | −0.69 | MMP7 | −0.73 | Resistin | −0.55 | CRP | −0.25 | | |
| 13 | 21.77 | IFNg | −0.18 | OPN | −0.74 | MMP7 | −0.76 | Resistin | −0.50 | CRP | −0.16 |
| 14 | 11.41 | IFNg | −0.35 | OPN | −0.82 | MMP7 | −0.22 | Resistin | −0.53 | ACRP30 | 0.33 |

| | | | | | CUTOFF VALUES | | | |
|---|---|---|---|---|---|---|---|---|
| | | | | SP | 85% | 90% | 95% | 99% |
| 1 | OPN | Resistin | | | 0.29 | 0.34 | 0.47 | 0.90 |
| 2 | IL1b | OPN | | | 0.85 | 1.03 | 1.39 | 1.80 |
| 3 | IFNg | OPN | | | 0.45 | 0.60 | 0.97 | 1.45 |
| 4 | OPN | MPO | | | 0.20 | 0.35 | 0.57 | 0.77 |
| 5 | OPN | VCAM | Resistin | | 0.21 | 0.33 | 0.44 | 0.84 |
| 6 | OPN | Fibrinogen | Resistin | | 0.29 | 0.40 | 0.50 | 0.73 |
| 7 | OPN | MMP7 | Resistin | | 0.25 | 0.40 | 0.49 | 0.86 |

| 8 | OPN | Resistin | APO-B100 | | | 0.20 | 0.26 | 0.35 | 0.80 |
|---|---|---|---|---|---|---|---|---|---|
| 9 | OPN | MMP7 | VCAM | Resistin | | 0.22 | 0.31 | 0.41 | 0.81 |
| 10 | IFNg | OPN | MMP7 | MPO | | 0.20 | 0.44 | 0.56 | 1.15 |
| 11 | IFNg | OPN | MMP7 | Resistin | | 0.22 | 0.38 | 0.58 | 0.89 |
| 12 | OPN | MMP7 | Resistin | CRP | | 0.25 | 0.36 | 0.56 | 0.88 |
| 13 | IFNg | OPN | MMP7 | Resistin | CRP | 0.26 | 0.36 | 0.47 | 1.06 |
| 14 | IFNg | OPN | MMP7 | Resistin | ACRP30 | 0.15 | 0.31 | 0.61 | 1.08 |

In TABLE 2, The columns in the top table component provide the offset values and the coefficient components derived by the scoring function algorithm to classify serum samples using individual marker panels. The bottom table provides the cutoff values for determination of N versus P classification at variable specificities for P ranging from 85% to 99%. At higher SP, fewer N samples will be classified while diminishing P values will be misclassified (from 15% to 1%).

An example of processing concentration data to obtain a score with 95% sensitivity for classification of P is provided in TABLE 3 for the five biomarker signature labeled number 13 in TABLE 2. The rows labeled N0010 and P0088 are serum sample concentration values obtained for two representative patients with the five measured values in picogram/ml for IFNg, OPN, MMP7, Resistin and CRP. Yellow highlights indicate the values used in the scoring algorithm.

TABLE 3

| | | Low |
|---|---|---|
| Marker | Coef | Cutoff |
| IFNg | −0.18 | 0.4123 |
| OPN | −0.74 | 3533 |
| MMP7 | −0.76 | 519.89 |
| Resistin | −0.5 | 9378.3 |
| CRP | −0.16 | 51754 |
| Offset: | 21.77 | |
| Cutoff: | 0.466 | |

| | | | | | | LN | LN | LN | LN | LN | | |
|---|---|---|---|---|---|---|---|---|---|---|---|---|
| | | | | | | (IFNg + | (OPN + | (MMP7 + | (resistin + | (CRP + | | |
| | IFNg | OPN | MMP7 | Resistin | CRP | Cut) | Cut) | Cut) | Cut) | Cut) | Score | DIAGN |
| Low CutOff | 0.41 | 3533.04 | 519.89 | 9378.30 | 51753.58 | | | | | | | |
| N0010 | 0.4 | 8317.9 | 4234.6 | 62707.3 | 65147.3 | −0.208 | 9.3802 | 8.4668 | 11.185 | 11.67 | 0.972 | N |
| P0088 | 5 | 4925.5 | 5599.3 | 296331 | 2062691 | 1.689 | 9.0429 | 8.7192 | 12.630 | 14.56 | −0.497 | P |

In TABLE 3, the five cells following the highlighted serum values for the row N0010 are values derived as the natural log (LN) of the sum of the concentration and the low cutoff value for each biomarker [e.g. LN (IFNg+Cut)=LN (0.40+0.41)=−0.208]. These values are serially derived for each marker and its low cutoff value. The overall score is then derived by adding the offset value to the sum of the products of the coefficient for each marker times the natural log calculation that was previously derived for each marker and its cutoff value.

For the examples provided in Table 3 and the values for patient N0010:

### Score=21.77+(−0.18)(−0.208)+(−0.74)(9.3802)+(−0.76)(8.4668)+(−0.5)(11.185)+(−0.16) (11.67)

### Score=0.972

### Diagnostic Classification=N

For the values for patient P0088:

### Score=21.77+(−0.18)(1.689)+(−0.74)(9.0429)+(−0.76)(8.7192)+(−0.5)(12.630)+(−0.16) (14.56)

### Score=−0.497

### Diagnostic Classification=P

If the score is greater than the cutoff value for the biomarker signature (in this panel, the cutoff=0.466 at 95% sensitivity for P), then the sample is classified as an N in the Diagnosis column (DIAGN). If it is less than the cutoff, it is classified as P. Therefore, the diagnosis of N and P was obtained for the patient test sets confirming the clinical findings. Any set of the designated marker panels may be applied in this manner using a software program or macro-subroutine whereby a series of concentration values is entered into the biomarker entry columns and the subroutine is applied to the subsequent cells to obtain the final scoring value. If the marker panel consists of fewer than 5 entries, zero is entered into the extra positions. The subroutines used to compute the values obtained in TABLE 3 are provided below highlighted where the rows and columns have been labeled to designate coordinates for the programmed subroutines.

TABLE 3A

| | A | B | C | D | E | F | G | H | I | J | K | L | M |
|---|---|---|---|---|---|---|---|---|---|---|---|---|---|
| | | | Low | | | | | | | | | | |
| 1 | Marker | Coef | Cutoff | | | | | | | | | | |
| 2 | IFNg | −0.18 | 0.4123 | | | | | | | | | | |
| 3 | OPN | −0.74 | 3533 | | | | | | | | | | |
| 4 | MMP7 | −0.76 | 519.89 | | | | | | | | | | |
| 5 | Resistin | −0.5 | 9378.3 | | | | | | | | | | |
| 6 | CRP | −0.16 | 51754 | | | | | | | | | | |
| 7 | Offset: | 21.77 | | | | | | | | | | | |
| 8 | Cutoff: | 0.466 | | | | | | | | | | | |
| | | | | | | | LN | LN | LN | LN | LN | | |
| | | | | | | | (IFNg + | (OPN + | (MMP7 + | (resistin + | (CRP + | | |
| 9 | | IFNg | OPN | MMP7 | Resistin | CRP | Cut) | Cut) | Cut) | Cut) | Cut) | Score | DIA |
| 10 | Low | 0.41 | 3533.04 | 519.89 | 9378.30 | 51753.58 | | | | | | | |
| | CutOff | | | | | | | | | | | | |
| 11 | N0010 | 0.4 | 8317.9 | 4234.6 | 62707.3 | 65147.3 | −0.208 | 9.380 | 8.467 | 11.185 | 11.67 | 0.972 | N |

TABLE 3 is reproduced above as TABLE 3A showing highlights on the cells which contain macro-subroutines to implement the scoring function algorithm upon data entry. The cells below contain the underlying subroutines (highlighted) derived to calculate the values for the corresponding labeled entries in table 3. These subroutines translate the scoring function algorithm into a spreadsheet that automatically calculates a Score and diagnosis (DIA) upon entry of patient serum values. In this case, a score and diagnosis may be derived from a panel containing fewer than 5 of these specific markers by entering a zero value. The panel must be modified with the appropriate coefficients, offset and cutoffs defined by the algorithm for the testing and use of other panels containing different biomarkers (see TABLE 3B)

TABLE 3B

| | | LN | LN | |
|---|---|---|---|---|
| LN (IFNg + Cut) | LN (OPN + Cut) | (MMP7 + Cut) | (resistin + Cut) | LN (CRP + Cut) |
| =LN(B11 + B$10) | =LN(C11 + C$10) | =LN(D11 + D$10) | =LN(E11 + E$10) | =LN(F11 + F$10) |

Score

=$B$7 + $B$2 * G11 + $B$3 * H11 + $B$4 * I11 + $B$5 * J11 + $B$6 * K11

DIAGN

=IF(L11 > $B$8, “N”, “P”)

The sensitivity of each of the 14 panels was computed against the 239 samples by performing cross validation testing. This testing was performed by randomly excluding 20% of the cases, optimizing the scoring function for the remaining 80% of cases and then testing the SF against the excluded cases. This was repeated 100 times, the average ROC curve accumulated, the area under the curve computed, and the sensitivities at SP=90%, 95% and 98% determined (TABLE 4). The computed sensitivities are lower than projected for the entire data set because of the smaller numbers.

TABLE 4

| | | | | | Optimized for | | | | | |
|---|---|---|---|---|---|---|---|---|---|---|
| | | | | | SP = 85% | | | Cross Validation | | |
| | | | | | SP | SN | | Testing | | |
| | | | | | for P | for N | AUC | Sens90 | Sens95 | Sens98 |
| OPN | Resistin | | | | 94.9 | 48.5 | 0.83 | 0.53 | 0.44 | 0.20 |
| IL1b | OPN | | | | 94.9 | 44.6 | 0.80 | 0.52 | 0.39 | 0.30 |
| IFNg | OPN | | | | 94.2 | 38.6 | 0.81 | 0.49 | 0.32 | 0.29 |
| OPN | MPO | | | | 94.2 | 39.6 | 0.83 | 0.51 | 0.29 | 0.17 |
| OPN | VCAM | Resistin | | | 95.7 | 50.5 | 0.83 | 0.53 | 0.42 | 0.18 |
| OPN | Fibrinogen | Resistin | | | 94.9 | 53.5 | 0.82 | 0.49 | 0.37 | 0.18 |
| OPN | MMP7 | Resistin | | | 94.9 | 50.5 | 0.84 | 0.54 | 0.42 | 0.29 |
| OPN | Resistin | APO- | | | 95.7 | 48.5 | 0.83 | 0.49 | 0.41 | 0.17 |
| | | B100 | | | | | | | | |
| OPN | MMP7 | VCAM | Resistin | | 94.9 | 54.5 | 0.84 | 0.57 | 0.50 | 0.37 |
| IFNg | OPN | MMP7 | MPO | | 94.2 | 57.4 | 0.81 | 0.50 | 0.33 | 0.24 |
| IFNg | OPN | MMP7 | Resistin | | 93.5 | 56.4 | 0.85 | 0.60 | 0.50 | 0.36 |
| OPN | MMP7 | Resistin | CRP | | 93.5 | 49.5 | 0.84 | 0.54 | 0.43 | 0.26 |
| IFNg | OPN | MMP7 | Resistin | CRP | 94.2 | 61.4 | 0.83 | 0.59 | 0.48 | 0.36 |
| IFNg | OPN | MMP7 | Resistin | ACRP30 | 94.2 | 58.4 | 0.82 | 0.60 | 0.45 | 0.25 |

In TABLE 4, the column SN for N indicates the percentage of the N samples that were correctly classified based on the scoring function algorithm optimized for a sensitivity of P sample classification at 85%. Classification of N was determined where P classification designated 95% of the samples correctly. The results obtained by cross validation against 20% of the sample population are provided in the last 3 columns. The labels sens90, sens95, and sens98 columns indicate the correctly classified cases for the N subjects while the SP for P was constant at 95%.

In conclusion, the unique scoring function algorithm coupled with serum assays of specific biomarker panels (14 different signatures comprising panels of 2 to 5 markers, 11 serum markers) provides a screening tool for identification of patients with coronary artery disease that will require percutaneous therapy. The algorithm has been translated into a macro-subroutine that can automatically compute scoring function values and a classification diagnosis upon data entry. The blood test will help identify those patients with minimal or no coronary artery disease that do not require treatment. Thus, the test will also help distinguish patients that do not require coronary angiography or electron beam CT scans of the coronary arteries to rule out the presence of CAD. The test provides this capability among patients presenting with symptoms associated with CAD in the emergency room or chest clinic. The efficacy of this test may extend to point-of-care identification of asymptomatic subjects with the potential for coronary artery disease among the general population. In that application, the test may have a higher positive predictive value than currently available through available blood or serum tests.

### 7. EXAMPLE

### Serum Protein Profiles Correlate with Coronary Artery Patency Among Patients Referred for Cardiac Catheterization

Background

Coronary heart disease affects 16.8 million patients annually in the United States at a cost of 165.4 billion dollars and has recently become the leading cause of death worldwide (Lloyd-Jones et al., 2010, Circulation 121(7):948-954). Referrals for coronary computed tomography (CT) angiography and cardiac catheterization are increasing annually despite potential morbidity, exposure to ionizing radiation and escalating costs. However, many patients clinically indicated for catheterization have minimal or no coronary artery disease (CAD) and require no subsequent therapeutic intervention. A noninvasive serum biomarker test to rule out catheterization among those patients referred for cardiac angiography but negative for CAD would have significant medical and economic value.

Methods:

We performed targeted serum proteomic profiling of 239 patients undergoing coronary angiography for evaluation of CAD. Among those patients, 138 underwent percutaneous intervention or coronary artery bypass graft surgery to treat hemodynamically significant CAD while the remaining 101 had neither significant disease (No CAD group) nor required follow-up treatment for CAD. The 2 groups were comparable in gender, BSA, cholesterol, LDL, diabetes and hypertension with a slight difference in age (No CAD: 58+/−12 vs CAD: 62+/−12 years, p<0.01) Thirty-three proteins were interrogated on a 56 sample training set and 24 analytes were validated on 239 samples using 2 multiplex antibody platforms.

Results:

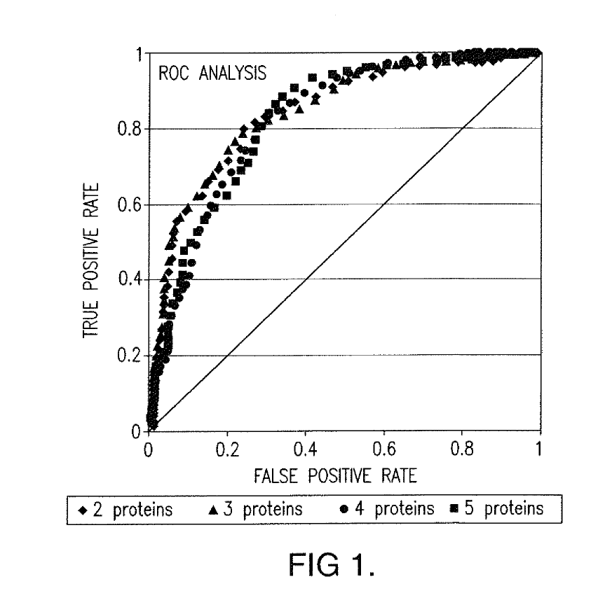

Highly significant increases were detected (false detection level=0.018) in serum of patients with confirmed CAD for Apo-B100, fibrinogen, VCAM-1, myeloperoxidase, CRP, resistin, osteopontin, IL-6, IL-1b, IL-10 and NT-pBNP. Apo-A1 fell significantly in this group. A predictive scoring algorithm was derived for classification of these patients (CAD vs. No CAD) using marker panels of 2 to 4 analytes. Discrimination of patients without CAD was achieved (86% Negative Predictive Value: NPV) using these biomarker panels while correctly classifying 95% (PPV) of the patients with CAD (see FIG. 1). Osteopontin, resistin, MMP7 and interferon-g were the most frequent classification biomarkers within the diagnostic panels.

Conclusions:

A serum biomarker test and scoring function algorithm has been developed with high predictive value among patients referred for cardiac catheterization. The test provides a novel evaluation criterion for patients regarding the need for diagnostic coronary angiographic studies and follow-up interventional therapy.

### 8. EXAMPLE

### Serum Proteomic Profiles Predict Angiographic Coronary Artery Disease in Symptomatic Patients Referred for Coronary Angiography

### 8.1 MATERIALS AND METHODS

Patient Group:

The patient samples comprised serum from 359 subjects referred for cardiac catheterization for symptoms associated with coronary artery disease. Blood was collected with patient informed consent according to an IRB approved genetic banking protocol (IRB #990835) after patient consent: 1) 15 ml of venous whole blood was drawn, 2) leukocyte centrifugation was performed immediately to pellet cells (300×g), 3) 250 ul to 2.0 ml of supernatant serum was transferred to a 1.5 ml cryotube, and 4) the samples were stored at −80° C. All 359 patients underwent diagnostic coronary angiography and 208 required interventional therapy i.e. percutaneous intervention (stent or angioplasty) or coronary artery bypass graft surgery (CABG) while the remaining patients did not have significant atherosclerosis that warranted coronary intervention. Thus, the serum samples derived from a single patient group based on clinical symptoms but represented two outcome groups based on the need for therapeutic intervention.

Proteomics Analysis:

Serum samples underwent a first thaw on ice to apportion them into 200 μl aliquots for processing. These aliquots were then stored at −80° C. until protein analysis was performed upon the next thaw cycle. All serum samples were processed in a randomized, blinded manner regarding patient characteristics and diagnostic classification. An exploratory study of 56 serum samples was performed using fluorokine multianalyte profiling (xMAP) of 33 analytes on the Luminex 100 platform (Luminex, Austin, Tex.) to determine serum dilution factors and to rule out targets lacking promise of statistical discrimination. The assay technology incorporated polystyrene microspheres dyed internally with differing ratios of two spectrally distinct fluorophores to create different spectrally addressed bead sets. Each bead set was conjugated with a biotinylated capture antibody specific for a target. The assays utilized a 96-well microplate format and were processed according to the manufacturer's protocol, including generation of a standard curve using recombinant target proteins over a four-fold dilution range. Standards and test samples were pipetted in duplicate at 25 μl per well and mixed with 25 μl of the bead mixture. Each microplate was incubated overnight at 4° C. on a microtiter shaker. Wells were washed with buffer (3 times) using a vacuum manifold for liquid removal and a secondary antibody was added to each well and incubated for 2 hours at room temperature. Streptavidin-PE was added to the wells and incubated for 30 minutes with constant agitation at room temperature. The wells were washed twice, assay buffer was added to each well, and samples were analyzed using the Bio-Plex suspension array system and Bio-Plex Manager software 4.0 (Bio-Rad Laboratories, Hercules, Calif.). Absolute quantities were determined by comparison to the 5 point standard curve for each analyte.

The Aushon-Searchlight Protein Array System (Aushon Biosystems, Inc, Billerica, Mass.) was used to interrogate all 359 unique patient serum samples in two different stages and analyte configurations (stage 1: 239 samples: 24 analytes; stage 2: 120 samples: 10 analytes). First, 239 samples were evaluated for 24 analytes over the concentration ranges defined by the preliminary study including the 56 samples analyzed previously. The assay comprised a multiplex sandwich ELISA using custom panels of monoclonal capture antibodies spotted in the wells of 96-well microtiter plates in a planar array. After serum incubation and washing, a second biotinylated monoclonal antibody targeted to a different site from the capture epitope was introduced for chemiluminescent signal detection of the serum analytes. The chemiluminescent reaction incorporated streptavidin-horseradish peroxidase (SA-HRP) that bound to the biotin site of the second antibody. Luminol Enhancer/Peroxidase solution was added and the HRP catalyzed oxidation of luminol to 3-aminophthalate resulted in emission of light at 428 nm. A chemiluminescent image was acquired by a cooled CCD 16-bit camera for processing by the SearchLight Array Analyst Software. The software employed a 4-parameter curve fit algorithm to calculate protein concentration of unknown samples. Calibration curves from recombinant protein targets in separate wells provided a reference to calculate absolute patient serum protein concentrations. Values for replicates of individual analytes, mean values, standard deviation, coefficient of variation, mean values adjusted for dilution and quality values were then derived. A curve fit quality program within the software was used to review the calibration and experimental data prior to reporting. This methodology resulted in detection of low abundance proteins at concentrations as low as 0.1 pg/ml.

The protein detection assays were performed in duplicate after dilution using a final volume of 50 pl. Quality control procedures included prescreening of multiplex panels for high sensitivity but minimal cross reactivity among target and detection antibodies. The largest panel provided quantitative data simultaneously on 7 analytes diluted 1:1 (volume/volume) (dilution factor: df=2×) in assay buffer (buffer: RPMI1640 w/o phenol red+10% heat inactivated FBS) including interferon-γ (IFNg), interleukin-1β (IL-1b), interleukin-6 (IL-6), interleukin-10 (IL-10), matrix metalloproteinase protein-1 (MMP-1), thrombomodulin (TM) and tumor necrosis factor-a (TNFa) (df=2×). Leptin, platelet endothelial cell adhesion molecule-1 (PECAM-1), endothelial leukocyte adhesion molecule-1 (E-Selectin), monocyte chemoattractant protein-1 (MCP-1), matrix metalloproteinase 7 and vascular cell adhesion molecule-1 (VCAM-1) were simultaneously assayed at a 25× dilution factor. Tissue inhibitor of metalloproteinase-1 (TIMP-1), fibrinogen, resistin, leukocyte selectin (L-Selectin) and myeloperoxidase (MPO) (df=1000×) were analyzed in a single panel of 5 analytes. Adiponectin (ACRP-30) and C-reactive protein (CRP) were assayed together at a serum dilution factor of 5,000×. Several analytes were interrogated independently including apolipoprotein-A1 (APO-A1, df=50,000×), apolipoprotein-B100 (APO B100, df=10,000×), osteopontin (OPN: df=10×) and N-terminal fragment protein precursor brain natriuretic peptide (NT-pBNP, df=2×).

A second stage study of 120 serum samples was performed to interrogate a subset of ten target analytes from among the initial 24 analytes included in the study of 239 serum samples but using additional samples. The sample prep, QC, methodological protocols for recombinant protein calibration profiles, serial dilutions and the serum assays were performed exactly as the previous study except for the use of fewer panels and different analyte configurations with a maximum multiplex configuration of 3 analytes per well. These assays provided quantitative data simultaneously on MPO, fibrinogen and resistin (df=1000×) evaluated in a 3-multiplex configuration. ACRP-30 and Apo-B100 analytes were evaluated in a multiplex configuration diluted in assay buffer (df=10,000×). Similarly, MMP-7 and VCAM-1 were evaluated together at a 25× dilution factor and IFNg and IL-1β were combined in a single panel at a lower dilution (df=2×). Osteopontin (OPN: df=10×) was assayed independently as before.

Statistical Analysis:

Patients were operationally defined as “symptomatic” by their referral for a clinically indicated catheterization. Based on the outcome of coronary angiography studies, the serum samples were derived from 150 patients with “normal” coronary arteries i.e. no significant coronary artery disease, while 209 serum specimens were derived from patients that had coronary disease requiring immediate therapy including stent placement, angioplasty or bypass graft surgery. The absolute concentrations of serum analytes in all 359 samples were derived via comparison to recombinant protein calibration curves run on the day the analysis was performed regardless of the different configurations of protein panels. The hypothesis subjected to statistical evaluation was that serum proteins were significantly different among the two patient endpoint classifications i.e. based on the requirement for clinical treatment of those patients with a diagnosis of CAD after coronary angiography. Statistical analysis was initially performed on the 239 samples in stage 1 evaluated for 24 analytes comprising 101 serum samples from patients with normal coronary arteries and 138 samples from patients requiring percutaneous intervention (PCI). These were the samples used to develop the scoring function algorithm. One hundred one samples from the second stage validation study interrogating 10 analytes were subsequently combined with the stage 1 results expanding the number of samples available for statistical comparison regarding those analytes. Statistical comparisons were performed to determine significant differences between patient groups among the 24 interrogated proteins and to separately assess the predictive strength of prospective protein signatures to classify the patient groups. The data were imported into Partek (Partek Genomics Suite, St. Louis, Mo.) for statistical comparison using the unpaired Students T test across the 2 patient groups for each analyte including calculation of a false detection rate (q value) to control for Type 1 errors arising from multiple tests.

Algorithm Development and Validation:

All 24 markers interrogated among the 239 serum samples in stage 1 were evaluated in multi-biomarker signatures to classify patients with CAD requiring treatment versus patients without clinically significant CAD as determined by coronary angiography and follow-up therapy. A scoring function (SF) algorithm was generated for all possible combination of proteins as disease “signatures” including an equivalent number of 24 “artificial” markers derived by randomly scrambling the data. The scoring function (SF) for each signature was constructed as a linear combination of natural logarithms of biomarker concentrations. Monte Carlo optimization was used to determine coefficients in the linear combination that provided highest diagnostic accuracy i.e. specificity (SP: identification of true negatives for significant CAD) for detecting patients with normal coronary arteries while maintaining 95% sensitivity (SN: identification of true positives for CAD requiring interventional therapy) for patients with coronary artery disease.

Each panel was evaluated for its specificity in discriminating serum samples derived from patients without coronary artery disease while maintaining 95% sensitivity in delineating samples from patients with coronary artery disease. An optimal scoring function was developed to obtain the highest detection rate for the normal patients.

The analytical form of the scoring function was:

S(φ=A₀+Σᵢ₌₁^{N}Aᵢ ln(Mᵢ(ρ)+Cᵢ) (1)

where Mᵢ(φ is the concentration of the i^{th} biomarker in the panel for participant ρ and A and C are numerical coefficients. Coefficient C can be selected to compensate for the effect of errors when a marker value is relatively small. Coefficient Cᵢ was selected to be 1/10th of the average value of the corresponding i^{th} biomarker. When score S(ρ) calculated for a participant ρ is positive, that participant is predicted to have CAD and when it is negative, that participant is predicted to be absent CAD. Optimization of the scoring function was performed to detect patients that did not have CAD. This involved Monte Carlo optimization using a Metropolis algorithm, to determine coefficients Ai where coefficient A₀ is selected to maintain 95% specificity for classification of patients with CAD. In order to estimate the performance of the scoring function, cross validation testing was performed using 80% of the data as the training data set and the remaining 20% of the data was used to test the specificity and sensitivity of the adjusted scoring function. All possible panels for the 24 markers were tested for their ability to discriminate between the two patient groups while successfully identifying 95% of the patients with coronary artery disease requiring percutaneous intervention.

Artificial marker values were generated and introduced into the data set to determine the effectiveness of the scoring function in delineating the best marker panels. The presence of an artificial marker in a top ranked panel indicated that the panel's performance was likely coincidental, as an artificial marker cannot predict the likelihood of the target disease. After ranking the performance of the panels via cross-validation analysis, those panels in the top of the final rankings without artificial markers were identified. The top 4 panels that did not include any artificial markers were identified for the 2, 3, and 4 marker panels, and the top 2 panels that did not include any artificial markers were identified for the 5-marker panel. The results are provided in TABLE 5.

TABLE 5
Top Ranked Panels Obtained by Cross-validation Testing. The numerical
values indicate the specificity (SP) of the top ranked panels to detect patients without
coronary artery disease at sensitivities (90%, 95%, 98%) indicated for the various panels
ranging from 2 to 5 markers. The results were determined at a sensitivity (SN) of ~95% to
correctly classify patients with coronary artery disease. AUC: area under ROC curve.
(n = 239 total, CAD = 138, no CAD = 101).

| | | | | | SN | AUC | SP90 | SP95 | SP98 |
|---|---|---|---|---|---|---|---|---|---|
| OPN | Resistin | | | | 94.2 | 0.84 | 0.548 | 0.393 | 0.117 |
| IL1b | OPN | | | | 94.2 | 0.82 | 0.449 | 0.304 | 0.217 |
| IFNg | OPN | | | | 94.9 | 0.77 | 0.379 | 0.314 | 0.247 |
| OPN | MPO | | | | 94.9 | 0.80 | 0.364 | 0.226 | 0.151 |
| OPN | VCAM | Resistin | | | 94.9 | 0.84 | 0.540 | 0.424 | 0.286 |
| OPN | Fibrinogen | Resistin | | | 94.9 | 0.83 | 0.500 | 0.365 | 0.237 |
| OPN | MMP7 | Resistin | | | 94.2 | 0.82 | 0.492 | 0.394 | 0.312 |
| OPN | Resistin | APO-B100 | | | 94.9 | 0.85 | 0.537 | 0.451 | 0.199 |
| OPN | MMP7 | VCAM | Resistin | | 95.7 | 0.82 | 0.533 | 0.436 | 0.326 |
| IFNg | OPN | MMP7 | MPO | | 95.7 | 0.84 | 0.669 | 0.584 | 0.278 |
| IFNg | OPN | MMP7 | Resistin | | 94.9 | 0.82 | 0.586 | 0.463 | 0.343 |
| OPN | MMP7 | Resistin | CRP | | 94.9 | 0.83 | 0.474 | 0.393 | 0.250 |
| IFNg | OPN | MMP7 | Resistin | CRP | 95.7 | 0.83 | 0.639 | 0.501 | 0.274 |
| IFNg | OPN | MMP7 | Resistin | ACRP30 | 94.9 | 0.82 | 0.635 | 0.499 | 0.304 |

We ranked >2 million combinations of 2, 3, 4 and 5 marker signatures comprising the 24 actual and 24 “artificial” markers for maximal ability to classify the patients in this study. For each signature from two to five markers, the top 50 panels with highest SP for normals—while correctly detecting at least 95% of the CAD patients—were re-examined using cross validation where 80% of participants were randomly selected as a training set to build the optimal SF and the remaining 20% of participants were then classified using this SF. The cross-validation procedure was repeated 500 times and the average SP and SN were used to identify the best performing signatures for detecting patients without significant coronary artery disease. The appearance of artificial (random) markers as components of signatures that provided best classification was taken as an indication that these panels included too many markers and differentiated among the groups based on chance variations.

Clinical validation of the scoring function algorithm was performed using the results obtained from an independent evaluation of 120 serum specimens for 10 of the markers that comprised the best biomarker signatures identified in the study of 239 patient samples. The 120 samples were obtained from symptomatic patients with clinical characteristics matching the previous 239 patients and the serum was collected according to the same protocol. The absolute concentration values obtained for these serum samples were entered into the algorithm in a macro-subroutine program using the offset, coefficients and cutoffs to detect CAD presence or absence based on patient outcome derived from the scoring function algorithm in the 239 patient study. The results of the 120 sample validation study were compared to the final diagnostic classification of each patient based on coronary catheterization and follow-up therapy to determine the sensitivity and specificity of each prospective diagnostic signature.

### 8.2 RESULTS

Diagnostic coronary angiography revealed that 209 of the patients in this study exhibited significant coronary artery disease mandating therapeutic intervention while 150 patients did not exhibit significant coronary artery disease despite symptoms or other findings that led to referral for cardiac catheterization. These 2 distinct outcome groups based on coronary angiography were otherwise identical upon admission regarding clinical symptoms and physical characteristics including gender, diabetic status, smoking history, diagnosis of hypertension, body surface area, basal metabolic rates, cholesterol, LDL and creatinine values (see TABLE 6). There were small albeit significant differences in age, HDL levels and ejection fraction between these groups but these differences were of minimal diagnostic value and all patients required coronary angiographic evaluation.

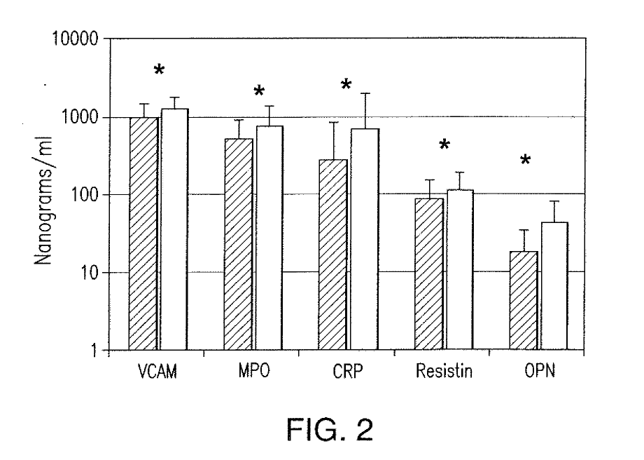

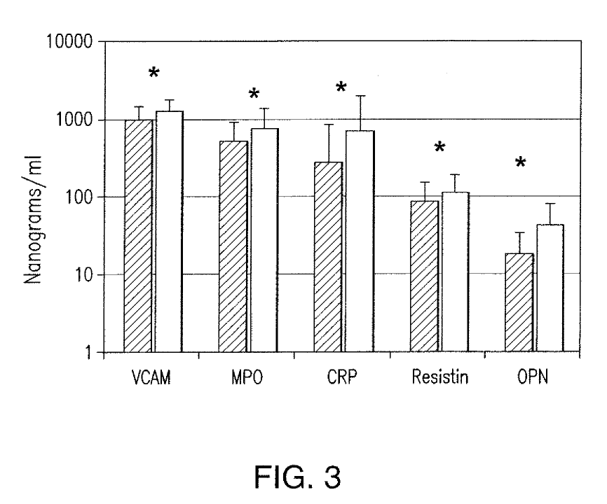

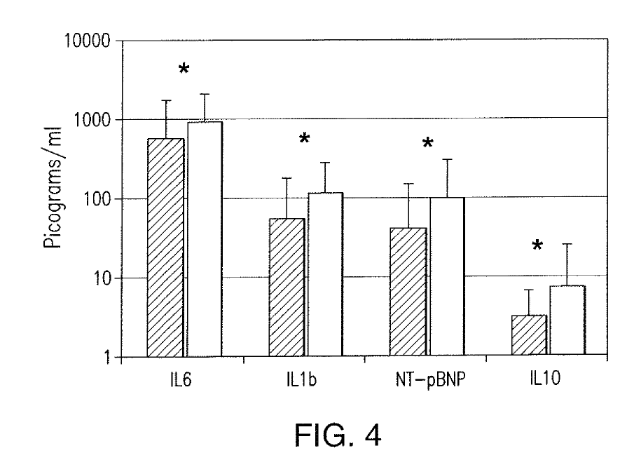

Significant differences were detected in 12 serum proteins (q value false detection rate=0.018) between samples from patients diagnosed with CAD requiring intervention and those with normal coronary arteries as determined by diagnostic coronary angiography. These differences were present in the stage 1 study of 239 patients and the results did not change with the addition of the 120 patients in the validation stage of the study (See TABLE 7). Apo-B100 and Apo-A1 were among the highest expressed proteins averaging approximately 300 μg/ml of serum (FIG. 2). Apo-B100 was significantly increased in patients with CAD versus normal coronary arteries while Apo-A1 fell significantly in the CAD group. Within the same dynamic concentration range, fibrinogen was present at levels typically exceeding a microgram per milliliter with values 4.6 fold higher in the patients with CAD (FIG. 2). At serum concentrations in a range from 1 nanogram to 1 microgram per milliliter of serum, 5 proteins were significantly higher in CAD patients. Specifically, VCAM-1, MPO, CRP, resistin and osteopontin were 1.3 to 2.5-fold higher than levels detected in normal patients (FIG. 3). Four analytes, IL-6, IL-1b, IL-10 and NT-pBNP were differentially expressed in a range from 1 picogram/ml to 1 nanogram/ml and all were significantly higher in the CAD group (FIG. 4).

Statistical analysis revealed that no other analytes from among the 41 interrogated targets using bead-based or planar platforms were significantly altered between the two groups of patient samples. For example, serum values for E-selectin, MMP-7, MCP-1, thrombomodulin and TIMP-1 yielded p values≧0.50 in comparisons between the two groups. Only MMP-1 levels approached statistical significance (No CAD: mean±S.D.=4.8±2.2 pg/ml, n=101 vs. CAD: 5.3±2.4 pg/ml, p=0.054, n=138) exhibiting a small increase (1.12 fold) in those patients with CAD.

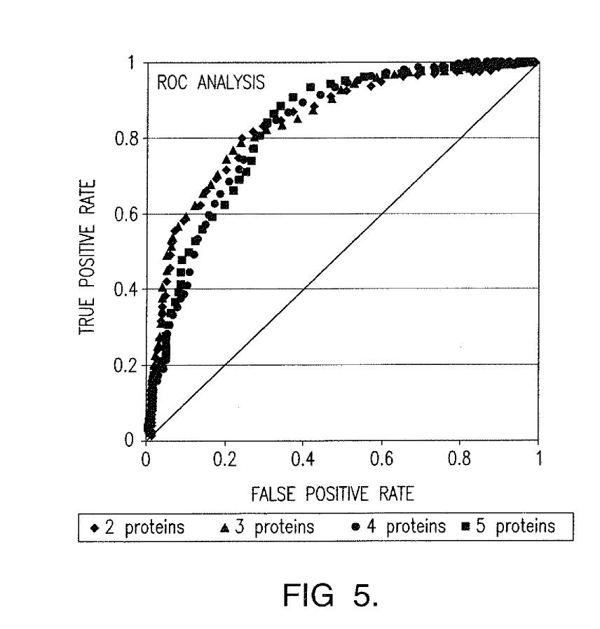

Predictive multimarker signatures were derived by testing all protein targets in the 239 sample study using a unique scoring function algorithm to discriminate CAD versus normal patients based on combinations of 2 to 5 biomarkers. We identified 14 signatures with highest acuity to detect patients with normal coronary arteries (42 to 65% SP) while detecting 95% of the CAD group (95% SN) (see Supplemental Methods-Panel Selection). Eleven proteins comprised the 14 signatures with osteopontin, resistin, MMP7, and IFNg most frequently represented. Receiver-operating characteristics analysis indicated that each of these signatures was similarly effective in discerning patients without CAD with minimal misclassification of CAD patients (5%). The area under the curve (AUC) for the top signatures ranged from 0.839±0.028 (mean±S.D.) for a 2 protein signature to a maximum AUC of 0.845 using 3 biomarkers (OPN, resistin, Apo B100) (FIG. 5). These ROC curves were compared to those generated by the Bayesian compound covariate predictor algorithm for the same data set. The area under the curve generated by the scoring function algorithm exceeded that obtained by the Bayesian predictor in every case. Subsequent clinical validation testing of 120 separate serum samples (49 normal, 71 patients requiring intervention) confirmed the first stage results. The serum signature with the highest ability to classify patient samples in the validation trial comprised osteopontin, resistin, MMP7, IFNg and ACRP 30. This signature successfully classified ≧92% of the patients with CAD while correctly delineating 35% of the patients that lacked significant CAD and therefore required no percutaneous coronary intervention or bypass graft surgery.

TABLE 6
Clinical characteristics of the patient groups.

| | AVE | | | AVE | | | |
|---|---|---|---|---|---|---|---|
| | NOR | S.D. | N | PCI | S.D. | N | P |
| AGE | 57.9 | 10.3 | 150 | 62.8 | 10.6 | 204 | 0.0006 |
| WT | 90.5 | 21.9 | 150 | 87.5 | 15.6 | 204 | NS |
| HT | 171.4 | 9.5 | 150 | 171.6 | 10.1 | 204 | NS |
| BSA | 2.0 | 0.3 | 150 | 2.0 | 0.2 | 204 | NS |
| BMI | 30.7 | 8.2 | 150 | 29.7 | 5.1 | 204 | NS |
| CHOL | 196.2 | 36.3 | 150 | 189.9 | 48.3 | 95 | NS |
| LDL | 120.9 | 37.8 | 134 | 119.4 | 42.5 | 90 | NS |
| HDL | 50.5 | 18.2 | 134 | 41.9 | 11.3 | 95 | 0.0001 |
| CREAT | 1.0 | 0.7 | 85 | 1.0 | 1.0 | 123 | NS |
| EF % | 57.9 | 8.4 | 148 | 53.5 | 10.8 | 106 | 0.0006 |
| GEND | 81 M | 69 F | 150 | 126 M | 78 F | 204 | |
| DIAB | 16 YES | 134 NO | 150 | 31 YES | 173 NO | 204 | |
| HYPTX | 76 YES | 74 NO | 150 | 125 YES | 79 NO | 204 | |

AVE (Average) and S.D. (standard deviations) for NOR patients (normal coronary arteries) and PCI patients (diagnosed with coronary artery disease requiring percutaneous therapeutic intervention).
N: number of samples for which parameters were available.
P: Statistical P values for individual parameters between the NOR and PCI groups. False detection rate = 0.018
NS indicates that no significant statistical differences were detected.
M/F indicates the number of males and females in each group with age is expressed in years, Wt (weight) in kilograms and HT (height) in centimeters.
BSA: body surface area calculated in meters2
BMI: body mass index = wt in kg/ht in meters2
CHOL: cholesterol,
LDL: low density lipoprotein,
HDL: high density lipoprotein and CREAT (creatinine) are reported in mg/dl (milligrams/decliter)
DIAB: (diabetes) and HYPTX (hypertension) reported as the number of positive (YES) or negative (NO) patients per group.

TABLE 7
Multiplex Proteomics Analysis of the CAD and Normal Patient Groups.

| | AVE NOR | S.D. | N | AVE PCI | S.D. | N | P value |
|---|---|---|---|---|---|---|---|
| IL-10 | 3.2 | 3.6 | 101 | 7.5 | 18.2 | 138 | 0.018 |
| IFNg | 3.5 | 10.7 | 150 | 4.8 | 9.5 | 204 | NS |
| TNFa | 22.4 | 73.7 | 101 | 15 | 19.4 | 138 | NS |
| NT-pBNP | 41.1 | 111.6 | 101 | 101.7 | 202.5 | 138 | 0.007 |
| IL-1b | 55.2 | 123.7 | 150 | 109.9 | 164 | 204 | 0.0006 |
| IL-6 | 578 | 1140 | 101 | 942 | 1154 | 138 | 0.016 |
| TM | 1444 | 859 | 101 | 1392 | 444 | 138 | NS |
| MCP-1 | 3069 | 3525 | 101 | 3347 | 3419 | 138 | NS |
| MMP-1 | 4751 | 2221 | 101 | 5342 | 2405 | 138 | NS |
| MMP-7 | 5523 | 3943 | 150 | 5712 | 2561 | 204 | NS |
| OPN | 17784 | 16543 | 150 | 41871 | 37825 | 204 | 1.75E−12 |
| Leptin | 13941 | 17641 | 101 | 10612 | 15495 | 138 | NS |
| PECAM-1 | 32060 | 28995 | 101 | 35036 | 25334 | 138 | NS |
| E-Selectin | 33943 | 19094 | 101 | 34309 | 16525 | 138 | NS |
| Resistin | 87283 | 61810 | 150 | 113015 | 79080 | 204 | 0.002 |
| CRP | 275344 | 550939 | 101 | 698542 | 1242218 | 138 | 0.002 |
| TIMP-1 | 320135 | 112187 | 101 | 329287 | 87929 | 138 | NS |
| MPO | 519904 | 380604 | 150 | 744547 | 601974 | 204 | 6.87E−05 |
| VCAM-1 | 973852 | 488016 | 150 | 1265187 | 501082 | 204 | 7.54E−08 |
| Fibrinogen | 4659675 | 5734653 | 150 | 21518046 | 56189244 | 204 | 0.0003 |
| L-Selectin | 1148218 | 281972 | 101 | 1112136 | 271368 | 138 | NS |
| Acrp30 | 6061321 | 4216767 | 150 | 5431911 | 4834458 | 204 | NS |
| Apo-A1 | 300607638 | 258848289 | 101 | 154165887 | 134757826 | 138 | 3.93E−08 |
| Apo-B100 | 290075527 | 88073018 | 150 | 316646861 | 89371624 | 204 | 0.005 |

Protein concentrations reported in picograms/ml.
ACRP-30: adiponectin,
APO-A1: apolipoprotein-A1,
APO B100: apolipoprotein-B100,
CRP: c-reactive protein,
E-Selectin: endothelial leukocyte adhesion molecule-1, fibrinogen,
IFNg: interferon-γ,
IL-1b: interleukin-1β,
IL-6: interleukin-6,
IL-10: interleukin-10, leptin,
L-Selectin: leukocyte selectin,
MCP-1: monocyte chemoattractant protein-1,
MMP-1: matrix metalloproteinase protein-1,
MMP-7: matrix metalloproteinase protein-7,
MPO: myeloperoxidase,
NT-pBNP: N-terminal fragment protein precursor brain natriuretic peptide,
OPN: osteopontin,
PECAM-1: platelet endothelial cell adhesion molecule-1, resistin,
TIMP-1: tissue inhibitor of metalloproteinase-1,
TM: thrombomodulin,
TNFa: tumor necrosis factor-α,
VCAM-1: vascular cell adhesion molecule-1.

### 8.3 DISCUSSION

Twelve proteins from among 41 analytes evaluated in this study were expressed at significantly different serum concentrations in patients with coronary artery disease requiring intervention versus symptomatic patients with no significant coronary disease based on cardiac catheterization studies. Proteins were selected for evaluation based on evidence that they contributed to important mechanisms underlying atherogenesis and plaque instability including vascular inflammation, aberrant lipid regulation, cell aggregation and/or vascular extracellular matrix (ECM) remodeling.¹² VCAM-1, IL-1b, IL-6, and IL-10 were significantly elevated in patients with CAD in the present study consistent with an injury-induced, inflammatory response.^{13,14} Elevated IL-1b and IL-6 have been associated previously with acute phase protein induction and may explain the concomitant increase in fibrinogen and CRP detected in the present study. Increased CRP has been considered a surrogate marker for inflammatory mediators in predicting coronary events along with NT-pBNP which was proposed as a marker of left ventricular dysfunction in CAD patient cohorts comparable to this study.^{10,14-16} CRP and NT-pBNP each significantly increased in association with CAD in the current study but were a weak classifier when combined compared to other duplex combinations. However, CRP was among the best single molecule classifiers delineating 19% of normal samples while detecting 95% of the CAD patients.

Significant reciprocal changes were detected in Apo-A1 and Apo-B (Apo-B100) in the CAD group in keeping with previous reports defining aberrant lipid transport and accumulation as a contributory factor to atherosclerosis.¹⁷ In fact, mutations in the Apo-B100 gene are known to cause autosomal dominant, hereditary familial hypercholesterolemia and premature coronary artery disease.¹⁸ Myeloperoxidase was also significantly increased in CAD patients associated with its role as a catalyst for lipid peroxidation at inflammation sites and a marker of plaque instability.^{19,20} Serum resistin concentrations were significantly increased in this study and have been previously correlated with metabolic shifts in lipid utilization as well as increased levels of proinflammatory cytokines including IL-1β, IL-6 and VCAM.²¹ Thus, proteins elevated in the CAD group in the present study reflected both ongoing inflammatory processes and altered lipid accumulation. In contrast, none of the targets traditionally associated with vascular smooth muscle and ECM remodeling were significantly altered among our patient groups including matrix metalloproteinases (MMPs) 1, 2, 3, 7, 9 and tissue inhibitors of metalloproteinases (TIMPs) 1, 2, 3 and 4. Only osteopontin, which acts as a negative regulator of calcification in bone remodeling, fit into this group with the rejoinder that it also may function as a chemokine regulator of inflammatory cell accumulation.²²

Multiplex proteomics analyses using precisely selected monoclonal antibodies have evolved to where these assays may be used systematically to analyze clinically relevant serum analytes by comparison to recombinant standards. Advantages of this approach include a requirement for small serum volumes (<100 μl) collected according to standard clinical protocols, rapid turnaround times (minutes to hours), high sensitivity and a broad dynamic range. Disadvantages include the high cost of the assays and poor concurrence of quantitative results across platforms associated with variations in antibodies, buffers and diluents. In the present study, 15 protein targets were tested at identical serum dilutions using both bead-based (Luminex) and planar (Aushon) technologies in 56 identical samples, albeit with different aliquots and in serial studies. Among these 15 comparisons, 12 assays concurred in detection of statistically significant differences among the 2 patient outcome groups despite the use of different reagents and technological platforms. These results indicated that multiplex immunochemical assays of serum may provide information of diagnostic relevance but that rigorous protocols and optimal reagents must be tested and standardized for clinical applications.

The results of this study were somewhat surprising both for the delineation of unexpected proteins as discriminants of CAD as well as the exclusion of several targets with established roles in the atherogenic process. For example, osteopontin was only indirectly associated with the process of atherogenesis yet exhibited the greatest statistical difference between patient groups and emerged most frequently among prospective biomarker signatures. Osteopontin was first identified as a sialoprotein from mineralized bone matrix and only recently has been associated with calcification of plaques in cardiac valves and vessels.^{23,24} Similarly, resistin was the second most frequent discriminant among diagnostic signatures using the scoring algorithm we developed and yet has been linked only indirectly to CAD through a role in metabolic homeostasis and insulin sensitivity.²⁵ These findings reinforce the idea that coronary artery disease is a pathological endpoint of diverse but converging physiological processes and that best performing diagnostic signatures reflect these disparities. However, the results were also notable for the large number of targets implicated in coronary artery disease that exhibited no significant differences between our patient cohorts including growth factors (VEGF, leptin, ghrelin), lipoproteins (Apo-A2, E, serum amyloid A: SAA), cell adhesion molecules (thrombospondin, PECAM-1, ICAM-1, selectins E, L, P) and the aforementioned MMP and TIMP targets associated with ECM remodeling. We cannot rule out degradation of various protein targets during storage as contributory to these results; however, all serum samples were collected, processed, stored and analyzed in an identical manner over the same timeframe. A more likely possibility was that our patients presented with relatively early stage coronary disease compared to other studies as evidenced by the fact that they had not yet suffered a myocardial “event”. A cohort with more advanced disease might reveal additional changes associated with larger or more unstable coronary plaque but at a potentially reduced efficacy of therapeutic intervention and greater risk to patient recovery.

The scoring function algorithm was developed, tested and validated using serum from patients with equivalent symptoms of coronary artery disease but with different therapeutic outcomes. Selection bias was avoided by testing a hypothesis driven biomarker panel and to avoid overfitting by performing cross-validation and follow-up testing of a separate patient cohort. All markers were tested for efficacy regardless of statistical significance in deriving predictive signatures and validation was performed with a separate but identical serum sample cohort.^{26,27}

### 8.4 REFERENCES

1. Lloyd-Jones D, Adams R J, Brown T M, Carnethon M, Dai S, De Simone G, Ferguson T B, Ford E, Furie K, Gillespie C, Go A, Greenlund K, Haase N, Hailpern S, Ho P M, Howard V, Kissela B, Kittner S, Lackland D, Lisabeth L, Marelli A, McDermott M M, Meigs J, Mozaffarian D, Mussolino M, Nichol G, Roger V L, Rosamond W, Sacco R, Sorlie P, Stafford R, Thom T, Wasserthiel-Smoller S, Wong N D, Wylie-Rosett J; American Heart Association Statistics Committee and Stroke Statistics Subcommittee. Executive summary: heart disease and stroke statistics—2010 update: a report from the American Heart Association. Circulation. 2010 Feb. 23; 121(7):948-54.
2. Xu J Q, Kochanek K D, Murphy S L, Tejada-Vera B. Deaths: Final data for 2007. National vital statistics reports; vol 58 no 19. Hyattsville, Md.: National Center for Health Statistics. 2010.
3. Scanlon P J, Faxon D P, Audet A M, Carabello B, Dehmer G J, Eagle K A, Legako R D, Leon D F, Murray J A, Nissen S E, Pepine C J, Watson R M, Ritchie J L, Gibbons R J, Cheitlin M D, Gardner T J, Garson A Jr, Russell R O Jr, Ryan T J, Smith S C Jr. ACC/AHA guidelines for coronary angiography. A report of the American College of Cardiology/American Heart Association Task Force on practice guidelines (Committee on Coronary Angiography). Developed in collaboration with the Society for Cardiac Angiography and Interventions. J Am Coil Cardiol. 1999 May; 33(6):1756-824.
4. Kolansky D M. Acute coronary syndromes: morbidity, mortality, and pharmacoeconomic burden. Am J Manag Care. 2009 March; 15(2 Suppl):S36-41.
5. Hacioglu Y, Gupta M, Budoff M J. Noninvasive anatomical coronary artery imaging versus myocardial perfusion imaging: which confers superior diagnostic and prognostic information? J. Comput Assist Tomogr 2010 34: 637-644.
6. Rosenberg S, Elashoff M R, Beineke P, Daniels S E, Wingrove J A, Tingley W G, Sager P T, Sehnert A J, Yau M, Kraus W E, Newby L K, Schwartz R S, Voros S, Ellis S G, Tahirkheli N, Waksman R, McPherson J, Lansky A, Winn M E, Schork N J, Topol E J; PREDICT (Personalized Risk Evaluation and Diagnosis in the Coronary Tree) Investigators. Multicenter validation of the diagnostic accuracy of a blood-based gene expression test for assessing obstructive coronary artery disease in nondiabetic patients. Ann Intern Med. 2010 Oct. 5; 153(7):425-34.
7. Fichtlscherer S, De Rosa S, Fox H, Schwietz T, Fischer A, Liebetrau C, Weber M, Hamm C W, Röxe T, Miller-Ardogan M, Bonauer A, Zeiher A M, Dimmeler S. Circulating microRNAs in patients with coronary artery disease. Circ Res. 2010 Sep. 3; 107(5):677-84. Epub 2010 Jul. 1.
8. Wang K, Zhang S, Weber J, Baxter D, Galas D J. Export of microRNAs and microRNA-protective protein by mammalian cells. Nucl. Acids Res. Online Jul. 7, 2010.
9. Katus H A, Remppis A, Neumann F J, Scheffold T, Diederich K W, Vinar G, Noe A, Matern G, Kuebler W. Diagnostic efficiency of troponin T measurements in acute myocardial infarction. Circulation. 1991 March; 83(3):902-12.
10. James S K, Lindahl B, Siegbahn A, Stridsberg M, Venge P, Armstrong P, Barnathan E S, Califf R, Topol E J, Simoons M L, Wallentin L. N-terminal pro-brain natriuretic peptide and other risk markers for the separate prediction of mortality and subsequent myocardial infarction in patients with unstable coronary artery disease: a Global Utilization of Strategies To Open occluded arteries (GUSTO)-IV substudy. Circulation. 2003 Jul. 22; 108(3):275-81. Epub 2003 Jul. 7.
11. Moriates C, Maisel A. The utility of biomarkers in sorting out the complex patient. Am J Med. 2010 May; 123(5):393-9.
12. Ross R. The pathogenesis of atherosclerosis: a perspective for the 1990s. Nature. Review. 1993 Apr. 29; 362(6423):801-9.
13. Erren M, Reinecke H, Junker R, Fobker M, Schulte H, Schurek J O, Kropf J, Kerber S, Breithardt G, Assmann G, Cullen P. Systemic inflammatory parameters in patients with atherosclerosis of the coronary and peripheral arteries. Arterioscler Thromb Vasc Biol. 1999 October; 19(10):2355-63.
14. Tedgui A, Mallat Z. Cytokines in atherosclerosis: pathogenic and regulatory pathways. Physiol Rev. 2006 April; 86(2):515-81.
15. Jernberg T, Stridsberg M, Venge P, Lindahl B. N-terminal pro brain natriuretic peptide on admission for early risk stratification of patients with chest pain and no ST-segment elevation. J Am Coil Cardiol. 2002 Aug. 7; 40(3):437-45.
16. Di Angelantonio E, Chowdhury R, Sarwar N, Ray K K, Gobin R, Saleheen D, Thompson A, Gudnason V, Sattar N, Danesh J. B-Type Natriuretic Peptides and Cardiovascular Risk. Systematic Review and Meta-Analysis of 40 Prospective Studies. Circulation. 2009 Dec. 1; 120(22):2177-2187.
17. Walldius G, Jungner I, Aastveit A H, Holme I, Curt D. Furberg C D, Sniderman A D. The apoB/apoA-I ratio is better than the cholesterol ratios to estimate the balance between plasma proatherogenic and antiatherogenic lipoproteins and to predict coronary risk. Clinical Chemistry and Laboratory Medicine 2004 42:12, 1355-1363.
18. Pullinger C R, Hennessy L K, Chatterton J E, Liu W, Love J A, Mendel C M, Frost P H, Malloy M J, Schumaker V N, Kane J P. Familial ligand-defective apolipoprotein B. Identification of a new mutation that decreases LDL receptor binding affinity. J Clin Invest. 1995 March; 95(3): 1225-34.
19. Zhang R, Brennan M L, Shen Z, MacPherson J C, Schmitt D, Molenda C E, Hazen S L. Myeloperoxidase functions as a major enzymatic catalyst for initiation of lipid peroxidation at sites of inflammation. J Biol Chem. 2002 Nov. 29; 277(48):46116-22. Epub 2002 Sep. 30.
20. Naruko T, Furukawa A, Yunoki K, Komatsu R, Nakagawa M, Matsumura Y, Shirai N, Sugioka K, Takagi M, Hozumi T, Itoh A, Haze K, Yoshiyama M, Becker A E, Ueda M. Increased expression and plasma levels of myeloperoxidase are closely related to the presence of angiographically-detected complex lesion morphology in unstable angina. Heart. 2010 November; 96(21):1716-22. Erratum in: Heart. 2010 December; 96(24):2044-5.
21. Stofkova A. Resistin and visfatin: regulators of insulin sensitivity, inflammation and immunity. Endocr Regul. 2010 January; 44(1):25-36.
22. Ashkar S, Weber G F, Panoutsakopoulou V, Sanchirico M E, Jansson M, Zawaideh S, et al. Eta-1 (osteopontin): an early component of type-1 (cell-mediated) immunity. Science 2000; 287:860-864.
23. Franzén A, Heinegard D. Isolation and characterization of two sialoproteins present only in bone calcified matrix. Biochem J. 1985 Dec. 15; 232(3):715-24.
24. Abdel-Azeez H A, Al-Zaky M. Plasma osteopontin as a predictor of coronary artery disease: association with echocardiographic characteristics of atherosclerosis. J Clin Lab Anal. 2010; 24(3):201-6.
25. Ye F, Than A, Zhao Y, Goh K H, Chen P. Vesicular storage, vesicle trafficking, and secretion of leptin and resistin: the similarities, differences, and interplays. J Endocrinol. 2010 July; 206(1):27-36. Epub 2010 Apr. 14.
26. Feng Z, Prentice R, Srivastava S. Research issues and strategies for genomic and proteomic biomarker discovery and validation: a statistical perspective. Pharmacogenomics. 2004 September; 5(6):709-19. Review.
27. Bossuyt P M, Reitsma J B, Bruns D E, Gatsonis C A, Glasziou P P, Irwig L M, Moher D, Rennie D, deVet H C W, Lijmer J G. The STARD statement for reporting studies of diagnostic accuracy: explanation and elaboration. Ann. Int. Medicine. 2003 January; 138 (1)40-44.

Various publications are cited herein, the contents of which are hereby incorporated by reference in their entireties.

1. A method of determining whether a patient is not likely to manifest significant coronary artery disease comprising:
(a) determining the serum levels of at least two biomarkers selected from the group consisting of osteopontin, resistin, interleukin 1β, interferon γ, myeloperoxidase, vascular cell adhesion molecule, fibrinogen, matrix metalloproteinase γ, apolipoprotein B100, C-reactive protein, and adipocyte complement related protein of 30 kDa;
(b) determining an Offset Value for the combination of biomarkers measured in (a);
(c) determining Coefficients for each of the biomarkers measured in (a);
(d) using the following scoring function to transform the serum levels determined in (a) into an IT score;
$$S ( p ) = {A}_{0} + {\sum }_{i = 1}^{N} {A}_{i} Ln ( {M}_{i} ( p ) + {C}_{i} )$$
where S(p) is the IT score for the patient p,
constant A₀ is the “Offset”
N is the number of biomarkers in the panel;
index i lists the markers in the panel
coefficient Aᵢ is the “Coefficient” for the i-th IT biomarker
Mᵢ(p) is the concentration in picograms/ml of the i-th IT biomarker for the patient p
Cᵢ is the “LowCutoff” value for the i-th biomarker;
(e) performing a cross-validation procedure, using a plurality of panels of biomarkers using a selected portion of the data set as a training data set to optimize values of Coefficients and LowCutoff values, for a predetermined number of times until an average specificity and sensitivity of the panel are evaluated to a desired accuracy, and
(f) comparing a final IT score to a Cutoff Score, wherein the final IT score is calculated using the optimized values of the Coefficients and LowCutoff values,
where an IT score greater than the Cutoff Score indicates that the patient is not likely to manifest significant coronary artery disease and an IT score less than the Cutoff Score indicates that the patient is likely to manifest significant coronary artery disease.

2. The method of claim 1, where the serum levels of osteopontin and resistin are measured.

3-20. (canceled)

21. The method of claim 1, wherein a value of N is selected to provide a predetermined level of specificity of the panel.

22. The method of claim 1, wherein the LowCutoff value for the i-th biomarker is selected to reduce volatility in the IT score at low biomarker values.
